(19) United States
(12) Patent Application Publication
(10) Pub. No.: US 2014/0234224 A1
(43) Pub. Date: Aug. 21, 2014

(54) COMPOSITIONS AND METHODS FOR MOLECULAR IMAGING OF OXYGEN METABOLISM

(71) Applicants: Pradeep M. Gupte, Airmont, NY (US); Robert L. DeLaPaz, Dobbs Ferry, NY (US); Ramanathan Ravichandran, Montebello, NY (US)

(72) Inventors: Pradeep M. Gupte, Airmont, NY (US); Robert L. DeLaPaz, Dobbs Ferry, NY (US); Ramanathan Ravichandran, Montebello, NY (US)

(73) Assignee: ROCKLAND TECHNIMED, LTD., Mahwah, NJ (US)

(21) Appl. No.: 14/346,920

(22) PCT Filed: Sep. 22, 2012
(86) PCT No.: PCT/US12/56775

§ 371 Date: Mar. 24, 2014

Related U.S. Application Data
(63) Continuation of application No. PCT/US12/36604, filed on May 4, 2012.
(60) Provisional application No. 61/537,823, filed on Sep. 22, 2011.

(51) Int. Cl. A61K 49/18

(52) U.S. Cl. CPC A61K 49/1806
USPC 424/9.37

(57) ABSTRACT

Provided are compositions containing an emulsion containing a perfluorinated compound, as well as methods for preparation of the compositions. Also provided are formulations containing a complex of oxygen-17 and the emulsion compositions. Additionally provided are methods for the preparation of the formulations as well as kits containing the formulations. Further provided are methods of use of the formulations in imaging of tissues using a magnetic resonance imaging system.

### CROSS-REFERENCES TO RELATED APPLICATIONS

This application claims priority to International Application No. PCT/US12/36604, filed May 4, 2012, which claims benefit of U.S. Provisional Application No. 61/537,823, filed Sep. 22, 2011, the disclosures of which are hereby incorporated by reference herein, in their entireties.

### BACKGROUND

Magnetic resonance imaging (MRI) systems rely on the tendency of atomic nuclei possessing magnetic moments to align their spins with an external magnetic field. Only nuclei with odd numbers of nucleons and non-integer spin have a magnetic moment, so only these nuclei can be detected and imaged. Hydrogen has one nucleon, a proton, in its nucleus and is the primary nucleus imaged at this time in medical practice.

The most common isotopes of oxygen, oxygen-16 and oxygen-18, occur naturally in air and have an even number of nucleons and hence, cannot be imaged in an MRI system. Oxygen-15 is an unstable (radioactive) isotope, produced in a cyclotron, that is used for positron emission tomography (PET) imaging and cannot be imaged with MRI. Oxygen-17 is a chemically identical, stable, non-radioactive oxygen isotope with the odd nucleon number and non-integer spin (5/2) necessary for magnetic resonance imaging. Oxygen-17 occurs naturally in air but in very low concentration (0.037 atm %) which has limited its use with MRI. Although Oxygen-17 gas (¹⁷O₂) can be concentrated as high as 70 atm % to 90 atm % and has been used in animal and human MRI studies by inhalation, the concentrating process is expensive and the volumes of gas needed for inhalation are quite high, making this method prohibitively expensive for widespread research or clinical use.

Fluorocarbon emulsions find uses as therapeutic and diagnostic agents. Most therapeutic uses of fluorocarbons are related to the remarkable oxygen-carrying capacity of these compounds. Perfluorocarbon emulsions using polysorbate surfactants may have a specific affinity to the endothelial cells of the blood brain barrier and can prodide a method of tissue specific drug delivery to the brain. Fluorocarbon emulsions have also been used in diagnostic imaging applications as a contrast agent by visualizing the fluorine distribution in tissue, including the focused distributions in targeted tissue such as the blood brain barrier of the central nervous system.

It is important that fluorocarbon emulsions intended for medical use exhibit particle size stability. Emulsions lacking substantial particle size stability are not suitable for long term storage, or they require storage in the frozen state. Emulsions with a short shelf life are undesirable. Storage of frozen emulsions is inconvenient. Further, frozen emulsions must be carefully thawed, reconstituted by admixing several preparations, then warmed prior to use, which is also inconvenient, and minor deviations in technique may result in an unusable emulsion.

Davis et al., (U.S. Pat. No. 4,859,363) describe stabilization of perfluorodecalin emulsion compositions by mixing a minor amount of a higher boiling point perfluorocarbon with the perfluorodecalin. Preferred higher boiling point fluorocarbons were perfluorinated saturated polycyclic compounds, such as perfluoroperhydrofluoranthene. Others have also utilized minor amounts of higher boiling point fluorocarbons to stabilize emulsions. (Meinert, U.S. Pat. No. 5,120,731 (fluorinated morpholine and piperidine derivatives), and Kabalnov, et al., Kolloidn Zh. 48: 27-32 (1986)(F-N-methylcyclohexylpiperidine)).

It has been suggested that a phenomenon responsible for instability of small particle size fluorocarbon emulsions is Ostwald ripening. During Ostwald ripening, an emulsion coarsens through migration of molecules of the discontinuous phase from smaller to larger droplets. (Kabalnov, et al., Adv. Colloid Interface Sci. 38: 62-97 (1992). The force driving Ostwald ripening appears to be related to differences in vapor pressures that exist between separate droplets. Such a difference in vapor pressure arises because smaller droplets have higher vapor pressures than do larger droplets. However, Ostwald ripening may only proceed where the perfluorocarbon molecules are capable of migrating through the continuous phase between droplets of the discontinuous phase. The Lifshits-Slezov equation relates Ostwald ripening directly to water solubility of the discontinuous phase. (Lifshits, et al., Soy. Phys. JETP 35: 331 (1959)).

### SUMMARY OF THE INVENTION

In certain aspects, this invention relates to compositions comprising an emulsion comprising a perfluorinated compound. Further aspects relate to methods for the preparation of the compositions. Additional aspects relate to formulations comprising a complex of oxygen-17 and the emulsion, methods for the preparation of the formulations, and kits comprising the formulations. Further aspects relate to methods of use of the formulations for imaging of tissues in a magnetic resonance imaging system.

### BRIEF DESCRIPTION OF THE DRAWINGS

FIG. 1 demonstrates O₂ adsorption in a perfluorcarbon emulsion under pressure.

FIG. 2 demonstrates O₂ release in a loaded perfluorcarbon emulsion after rapid pressure drop.

FIG. 3 shows a particle size distribution for a composition prepared according to Example 14. The composition was prepared using five (5) passes through a microfluidizer at 27,000 psi.

FIG. 4 shows a particle size distribution for a composition prepared according to Example 14. The composition was prepared using five (5) passes through a microfluidizer at 27,000 psi and autoclaved 1× at 121° C. for 15 minutes.

FIG. 5 shows a particle size distribution for a composition prepared according to Example 14. The composition was prepared using five (5) passes through a microfluidizer at 27,000 psi and autoclaved 2× at 121° C. for 15 minutes.

FIG. 6 shows a particle size distribution for a composition prepared according to Example 14. The composition was prepared using five (5) passes through a microfluidizer at 27,000 psi and autoclaved 3× at 121° C. for 15 minutes.

### DETAILED DESCRIPTION OF THE INVENTION

In certain aspects, the present invention relates to methods of ¹⁷O₂ delivery for MRI in animals and humans utilizing small volumes of gas on an oxygen-avid carrier (perfluorohydrocarbon emulsion) administered intravascularly.

The use of perfluorohydrocarbons as oxygen carrying blood substitutes is very beneficial, considering their efficiency in delivering oxygen to a target organ. Oxygen is highly soluble in liquid perfluoro-chemicals. In contrast, normal saline or blood plasma dissolves about 3% oxygen by volume, whole blood about 20%, whereas perfluorochemicals can dissolve up to 40% and more. However, even though the fluorochemicals have the ability to adsorb large quantities of oxygen, the intraveneous injection of non-emulsified perfluorochemicals can be highly toxic since they are immiscible with blood and can therefore produce emboli.

In certain aspects, the present invention relates methods for emulsifying a perfluorocarbon with an emulsifying agent to produce a synthetic oxygen carrier that meets criteria for use in physiological systems and to the compositions so produced. Preferably, the synthetic oxygen carrier produced in accordance with certain embodiments of the present invention may form a stable, fine emulsion that is non-toxic, non-mutagenic, and compatible with blood and endothelial cells, preferably having insignificant pharmacological, physiological, and biochemical activity, and is preferably excreted unchanged in physiological systems.

In certain aspects, methods are described for the use of multinuclear magnetic resonance imaging (¹H, ¹⁷O, ¹⁹F) after administrating an effective imaging amount of a diagnostic imaging agent comprising a complex of oxygen. The imaging agent is preferably comprised of a complex of the non-radioactive isotope, oxygen-17, and a biologically acceptable liquid carrier. Preferably, a biologically acceptable emulsifying agent is used. Preferably, the emulsifying agent may be used for biocompatibility and stability. Preferably, the complex has an ionic and osmotic composition essentially equal to that of blood.

As used herein, the term “perflourinated” refers to an organic structure where each of the hydrogen atoms attached to a carbon atom is replaced by fluorine.

A perfluorinated compound is preferred for use in an emulsion composition, although it is possible to use other liquids including blood or blood plasma. The perfluorinated compounds, however, have the ability, to adsorb large amounts of oxygen. As such, in a preferred embodiment, the perfluorinated compound may be selected from a group that includes, but is not limited to, perfluoro(tert-butylcyclohexane), perfluorodecalin, perfluoroisopropyldecalin, perfluoro-tripropylamine, perfluorotributylamine, perfluoro-methylcyclohexylpiperidine, perfluoro-octylbromide, perfluoro-decylbromide, perfluoro-dichlorooctane, perfluorohexane, dodecafluoropentane, perfluorodimethyladamantane, perfluorooctylbromide, perfluoro-4-methyl-octahydroquinolidizine, perfluoro-N-methyl-decahydroquinoline, F-methyl-1-oxa-decalin, perfluoro-bicyclo[5.3.0]decane, perfluorooctahydroquinolidizine, perfluoro-5,6-dihydro-5-decene, perfluoro-4,5-dihydro-4-octene and mixtures thereof. Preferably, the highly fluorinated organic compound is selected from perfluorodecalin, perfluorooctylbromide, perfluoro(tert-butylcyclohexane and mixtures thereof.

Accordingly, one embodiment of the present invention is directed to a fluorocarbon emulsion, comprising:

a continuous fluorocarbon immiscible hydrophilic liquid phase; and

a dispersed phase comprising fluorocarbon suspended as droplets within the continuous phase.

One embodiment of this invention relates to a composition comprising an emulsion, which comprises perfluorinated oxygen-avid compound particles and at least one emulsifying agent. Preferably, the emulsion is biocompatible. Preferably, the emulsion is bioinert.

In certain embodiments, there is provided a composition comprising an emulsion comprising particles of at least one perfluorocarbon and at least one emulsifying agent. In certain embodiments, the composition comprises two or more emulsifying agents. In certain embodiments, one or more of the emsulsifying agents may be a surfactant.

Preferrably, the particles have an effective average particle size of between about 0.1 μm and about 5 μm or between about 0.3 μm and about 1.5 μm. In certain embodiments, the particle size distribution has a z-average of equal to, or less than, about 0.3 μm. In certain embodiments, about 95% of the particles have an effective size of less than about 1.5 μm.

It is preferable for the effective particle size of the perfluorinated compound particles to be less than about 1.5 microns. In certain embodiments, this particle size may facilitate the transport of oxygen to abnormal target tissues with compressed, constricted or partially thrombosed microvasculature that may not be reached by red blood cells, which have a diameter of approximately 6-8 μm. In certain embodiments, this particle size in normal diameter capillaries, with normal or reduced flow, may improve the passage of oxygen from hemoglobin in red blood cells to tissue by providing a facilitated diffusion pathway or “oxygen diffusion bridge” with lower resistance to oxygen passage than normal blood plasma.

In certain embodiments, the perfluorinated compound is preferably present in an amount of about 5% to about 85% or from about 15% to about 70%, by weight of the composition. Preferably, the perfluorinated compound is present at about 50% (w/w). Preferably, the emulsifying agent is present in an amount from about 1% to about 20%, from about 1% to about 10%, from about 4% to about 8%, from about 4% to about 6%, from about 4% to about 7%, about 10%, about 9%, about 8%, about 7%, about 6%, about 5%, about 4%, about 3%, about 2% or about 1% by weight of the composition.

As used herein, the term “biocompatible” refers to a substance that does not produce an inflammatory, immune, chemical, toxic or other reaction in vivo. “Bioinert” refers to a substance that is biocompatible and excreted from the body while still intact.

In certain embodiments, the invention provides compositions comprising an emulsion wherein the emulsion comprises a first component comprising a highly fluorinated organic compound and a second component which may retard Ostwald ripening of the emulsion. Preferably, the emulsion is biocompatible. Preferably, the emulsion is bioinert. In certain embodiments, the second component is not substantially surface active. In certain embodiments, the second component is not significantly water soluble. In certain embodiments, the second component may comprise at least one second lipophilic fluorocarbon.

In certain embodiments, the second component is present in a quantity of from about 1% to about 15% of the total weight of the composition.

In certain embodiments, suitable second components or additives that may be used in the emulsions and processes of the invention include, but are not limited to, liquid fatty oils, hydrocarbons, waxes, such as monoesters of a fatty acid and a monohydroxide alcohol, long chain ethers, diglycerides, triglycerides silicone oils and nitriles. These include, without limitation, palmitoyl oleate, octyl nitrile, dodecyl nitrile, triglycerides of fatty acids such as soy oil, and safflower oil, hexadecane, diglycerides having a C₁₂₋₁₈ carbon chain and one unsaturation, and mineral oil. These oils also may be used singly or in various combinations in the emulsions and processes in various embodiments of the invention. When the emulsions are to be used medically, the oil or combination of oils must, of course, be physiologically acceptable. In certain embodiments, a second component that may be used to retard Ostwald ripening in the emulsions and processes of this invention include, for example, oils that are preferably not substantially surface active and not significantly water soluble.

In certain embodiments, the second component or additive may be selected from the group including, but not limited to: liquid fatty oils, hydrocarbons, waxes, such as monoesters of a fatty acid and a monohydroxide alcohol, long chain ethers, monoglycerides, diglycerides, triglycerides, vegetable oils, and mixtures thereof.

In certain embodiments, the amount of oil, or oils, present in the emulsions may vary over a wide range of concentrations. It depends on the concentration and properties of the other components of the emulsion, being principally dependent on the characteristics of the fluorocarbon component of the emulsion. The actual oil concentration to produce an acceptable emulsion for any given set of components may be determined using techniques of preparing and testing the stability of emulsions at various oil concentrations.

In certain embodiments, the second component or additive may be selected from the group including, but not limited to, safflower oil, soybean oil, sunflower oil, ricinus oil and mixtures thereof. Preferably, the second component may be present in the composition in the range of about 1% to about 10%, about 1% to about 5%, about 1% to about 2%, about 10%, about 9%, about 8%, about 6%, about 5%, about 4%, about 3%, about 2% or about 1% by weight of the composition.

In certain embodiments, the second component is a lipophilic fluorocarbon moiety.

In certain embodiments, there is provided a composition comprising an emulsion, the emulsion comprising a continuous aqueous phase, and a discontinuous fluorocarbon phase. In certain embodiments, the emulsion comprises a one or more first fluorocarbon, and a one or more second fluorocarbon having a molecular weight greater than each such first fluorocarbon. In certain embodiments, the emulsion comprises from about 50% to about 99.9% of a one or more first fluorocarbons, and from about 0.1% to about 50% of one or more second fluorocarbons having a molecular weight greater than each such first fluorocarbon. Preferably, each such second fluorocarbon includes at least one lipophilic moiety. The first fluorocarbon can be selected from a variety of materials, including, but not limited to, perfluorobutyltetrahydrofuran, perfluoro-n-octane, perfluoropolyether, perfluoromethyldecalin, perfluororcyclohexyldiethylamine, perfluoro-isopentylpyran, perfluorodibutylmethylamine, perfluoro(tert-butylcyclohexane), perfluorodecalin, perfluoroisopropyldecalin, perfluoro-tripropylamine, perfluorotributylamine, perfluoro-methylcyclohexylpiperidine, perfluoro-octylbromide, perfluoro-decylbromide, perfluoro-dichlorooctane, perfluorohexane, dodecafluoropentane, or a mixture thereof, perfluorodimethyladamantane, perfluorooctylbromide, perfluoro-4-methyl-octahydroquinolidizine, perfluoro-N-methyl-decahydroquinoline, F-methyl-1-oxa-decalin, perfluoro-bicyclo[5.3.0]decane, perfluorooctahydroquinolidizine, perfluoro-5,6-dihydro-5-decene, perfluoro-4,5-dihydro-4-octene and mixtures thereof. Preferably, the highly fluorinated organic compound is selected from perfluorodecalin, perfluorooctylbromide, perfluoro(tert-butylcyclohexane) and mixtures thereof.

In certain embodiments, the first highly fluorinated organic compound is present in the emulsion in an amount between about 20% and about 60% by weight, or between about 30% and about 55% by weight, or in an amount of about 50% by weight of the emulsion.

In certain embodiments, in the second fluorocarbon, the lipophilic moiety or moities may be, without limitation, Br, Cl, I, H, CH₃, substituted on a saturated or unsaturated hydrocarbon. In one embodiment, the second fluorocarbon is an aliphatic perfluorocarbon having the general formula CₙF₂ₙ₊₁R or CₙF₂ₙR₂, wherein n is an integer from 9 to 12 and R is the lipophilic moiety. In various embodiments, the second component is selected from the group including, but not limited to, perfluorododecyl bromide, C₁₀F₂₁CH═CH₂, C₁₀F₂]CH₂CH₃, linear or branched brominated perfluorinated alkyl ethers and mixtures thereof. Preferably, the second fluorocarbon comprises perfluorodecyl bromide. In certain embodiments, the discontinuous fluorocarbon phase of the emulsion comprises from about 60% to about 99.5% of the first fluorocarbon, and from about 0.5% to about 40% of the second fluorocarbon; or from about 80% to about 99% of the first fluorocarbon, and from about 1% to about 20% of the second fluorocarbon.

In certain embodiments, the emulsion comprises an emulsifying agent. In certain embodiments, the emulsion comprises a stabilizing agent, wherein the stabilizing agent reduces the ability of the fluorocarbon droplets to move within the continuous phase.

Without intending to be bound by any theory of operation, the fluorocarbon emulsion may be stabilized by further decreasing the ability of the dispersed fluorocarbon droplets to move within the continuous phase. This result may achieved by several means including, but not limited to, using a stabilizing agent to alter the physical properties of the continuous phase, an emulsifying agent, and/or a method of making the emulsion that results in a highly stabilized fluorocarbon emulsion.

The stabilizing agent may be selected from a group including, but not limited to, cetyl alcohol, stearyl alcohol, behenyl alcohol, glyceryl stearate, polyoxyethylated fatty acid (PEG-75 stearate), polyethylene glycol ether of cetyl alcohol (ceteth-20), polyethylene glycol ether of stearyl alcohol (steareth-20), hydrogenated phosphotidylcholine, and mixtures thereof. In certain embodiments, the amount of the stabilizing agent may be in the range from about 0.05% to about 10% (wt/wt). In another embodiment, both the stabilizing agent and the emulsifying agent may be the same compound.

The emulsifying agent included in the composition can be selected from a wide variety of commercially available products. The particular agent chosen will preferably be one which is non-toxic, biologically acceptable, compatible with both the oxygen-17 and the perfluorinated compound, and have no adverse effects on the body. It has been observed that the known family of polyoxyethyenepolyoxypropylene copolymers not only emulsify the organic phase, but can also serve as a plasma expander to reproduce the oncotic pressure normally provided by blood proteins. These polyols are nontoxic at low concentrations and unlike many ionic and non-ionic surfactants, they do not cause hemolysis of erythrocytes.

In certain embodiments, there is provided a composition comprising an emulsion comprising particles of at least one perfluorocarbon and at least one emulsifying agent. In certain embodiments, an emulsifying agent may be a surfactant. In certain embodiments, the emulsion may comprise one or more surfactants. In certain embodiments, the composition comprises one or more surfactants in a total amount of from about 1% to about 10%, from about 4% to about 8%, from about 4% to about 7%, from about 4% to about 6%, about 10%, about 9%, about 8%, about 7%, about 6%, about 5%, about 4%, about 3%, about 2%, or about 1% by weight of the composition.

In certain embodiments, the amounts of a second component and/or surfactant in the emulsion are dependent on the volume percent of highly fluorinated organic compound and are preferably present in amounts effective to produce emulsions according to aspects of the invention.

In certain embodiments, use of a surfactant comprising a phospholipid is preferred. In certain embodiments, an emulsifying agent may be a surfactant that may be prepared from naturally occurring precursor materials such as lecithin, from a synthesized counterpart of lecithin-derived materials, or from any other material known to those in the art. In one embodiment, the emulsifying agent is a surfactant selected from a group that includes, but is not limited to, soy lecithin, phosphatidyl choline, phosphatidyl inositol, and phosphatidylethanolamine and mixtures thereof. In a preferred embodiment, the surfactant may be purified from soy lecithin. Soy lecithin is a complex mixture of phospholipids, glycolipids, triglycerides, sterols, and small quantities of fatty acids, carbohydrates, and sphingolipids. The primary phospholipid components of soy lecithin include phosphatidyl choline (13-18%), phosphatidylethanolamine (10-15%), phosphatidyl inositol (10-15%), phosphatidic acid (5-12%).

In certain embodiments, surfactant may be selected from a group including, but not limited to, egg yolk phospholipids, soya phospholipids, hydrogenated phosphatidylcholine, lysophosphatidylcholine, phosphatidylethanolamine, phosphatidylserine, phosphatidylinositol, phosphanolipids, phosphatidic acid, and mixtures thereof.

In certain embodiments, preferred surfactants include: egg phospholipids with 80% phosphatidylcholine (E-80, available from Lipoid), egg phospholipids with 70% phosphatidylcholine (E-80S, available from Lipoid), fatfree soybean phospholipids with 70% phosphatidylcholine (S75, available from Lipoid), and mixtures thereof. Preferably, the composition comprises a phospholipid surfactant in an amount of from about 1% to about 10%, from about 4% to about 6%, about 10%, about 9%, about 8%, about 6%, about 5%, about 6%, about 5%, about 4%, about 3%, about 2% or about 1% by weight of the composition.

Additionally, among the surfactants useful in the emulsions of this invention are any of the known anionic, cationic, nonionic and zwitterionic surfactants. These include, for example, anionic surfactants, such as alkyl or aryl sulfates, sulfonates, carboxylates or phosphates, cationic surfactants such as mono-, di-, tri-, and tetraalkyl or aryl ammonium salts, nonionic surfactants, such as alkyl or aryl compounds, whose hydrophilic part consists of polyoxyethylene chains, sugar molecules, polyalcohol derivatives or other hydrophilic groups and zwitterionic surfactants that may be combinations of the above anionic or cationic groups, and whose hydrophobic part consists of any other polymer, such as polyisobutylene or polypropylene oxides.

In certain embodiments, useful surfactants may include polysorbates, including, but not limited to, polysorbate 20, polysorbate 40, polysorbate 60, polysorbate 80 (Tween® 20, 40, 60, or 80) or mixtures thereof. Preferably, the composition comprises from about 0.5% to about 2.5%, from about 1% to about 2.5%, from about 1.5% to about 2.5%, from about 2% to about 2.5%, from about 1.5% to about 2%, from about 1% to about 2%, about 2.5%, about 2.4%, about 2.3%, about 2.2%, about 2.1%, about 2%, about 1.5%, about 1.0% or about 0.5% polysorbate surfactant by weight of the composition.

In certain embodiments, the emulsifying agent is a non-fluorinated compound. In one embodiment, the non-fluorinated emulsifying agent is a hydrogenated phospholipid. The hydrogenated phospholipid may be selected from the group consisting of hydrogenated phosphatidylcholine, lysophosphatidylcholine, phosphatidylethanolamine, phosphatidylserine, phosphatidylinositol, phosphanolipids, phosphatidic acid, and mixtures thereof.

In certain embodiments, combinations of surfactants may be used in the emulsions of this invention. In addition, mixtures of compounds, one or more of which are not surfactants, but which compounds when combined act as surfactants may also be usefully employed as the surfactant component of the emulsion.

In certain embodiments, the composition comprises at least one further component or additive selected from among liquid fatty oils, hydrocarbons, waxes, such as monoesters of a fatty acid and a monohydroxide alcohol, long chain ethers, diglycerides, triglycerides silicone oils and nitriles. These include, for example, palmitoyl oleate, octyl nitrile, dodecyl nitrile, triglycerides of fatty acids such as soy oil, and safflower oil, hexadecane, diglycerides having a C₁₂₋₁₈ carbon chain and one unsaturation, and mineral oil. In certain embodiments, this further component may be used to retard Ostwald ripening in the emulsion. Such a component may include, for example, one or more oils that are preferably not substantially surface active. Preferably, the component is not not significantly water soluble.

In certain embodiments, this component or additive may be selected from the group including, but not limited to: liquid fatty oils, hydrocarbons, waxes, such as monoesters of a fatty acid and a monohydroxide alcohol, long chain ethers, monoglycerides, diglycerides, triglycerides, vegetable oils, and mixtures thereof.

In certain embodiments, the amount of oil, or oils, present in the emulsions may vary over a wide range of concentrations. It depends on the concentration and properties of the other components of the emulsion, being principally dependent on the characteristics of the fluorocarbon component of the emulsion. The actual oil concentration to produce an acceptable emulsion for any given set of components may be determined using techniques of preparing and testing the stability of emulsions at various oil concentrations.

In certain embodiments, this component or additive may be selected from the group including, but not limited to, safflower oil, soybean oil, sunflower oil, ricinus oil and mixtures thereof. Preferably, this component may be present in the composition in the range of about 1% to about 10%, about 1% to about 5%, about 1% to about 2%, about 10%, about 9%, about 8%, about 7%, about 6%, about 5%, about 4%, about 3%, about 2% or about 1% by weight of the composition.

In certain embodiments, emulsions according to the invention may also contain other components conventionally used in “artificial bloods” or blood substitutes, oxygen transport agents or contrast agents for biological imaging. For example, in certain embodiments, the emulsion may contain an isotonic agent, to adjust the osmotic pressure of the emulsion to about that of blood. Exemplary agents include, but are not limited to, glycerol and sodium chloride (NaCl). In certain embodiments, agents may be added to the emulsion to adjust osmolarity to the approximate physiological value of about 300 mOsm/l with a range of from about 290-600 mOsm/l. Preferably, amounts may be added as needed to reach target osmolarity. However, other amounts and other osmotic pressure controlling agents, e.g., Tyrode solution, could as well be used. The emulsions of this invention may also include other components, such as, without limitation, oncotic agents, e.g., dextran or HES, and antioxidants.

In certain embodiments, the perfluorocarbon employed in the compositions and methods described herein may be in compositions which may further comprise pharmaceutically acceptable carrier or cosmetic carrier and adjuvant(s) suitable for intravenous, intra-arterial, intravascular, intrathecal, intratracheal or topical administration. Compositions suitable for these modes of administration are well known in the pharmaceutical and cosmetic arts. These compositions can be adapted to comprise the perfluorocarbon or oxygenated perfluorocarbon. The compositions employed in the methods described herein may also comprise a pharmaceutically acceptable additive.

The compositions disclosed herein may comprise excipients such as solubility-altering agents (e.g., ethanol, propylene glycol and sucrose) and polymers (e.g., polycaprylactones and PLGA's) as well as pharmaceutically active compounds. In certain embodiments, the compositions may contain antibacterial agents which are non-injurious in use, for example, without limitation, thimerosal, benzalkonium chloride, methyl and propyl paraben, benzyldodecinium bromide, benzyl alcohol, or phenylethanol.

In certain embodiments, the compositions may also contain one or more buffering ingredients such as, without limitation, sodium acetate, gluconate buffers, phosphates, bicarbonate, citrate, borate, ACES, BES, BICINE, BIS-Tris, BIS-Tris Propane, HEPES, HEPPS, imidizole, MES, MOPS, PIPES, TAPS, TES, Tricine or glycine.

In certain embodiments, the compositions may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, or ethylenediamine tetraacetic acid. In certain embodiments, the composition comprises ethylenediaminetetraacetic acid (EDTA) disodium dihydrate, preferably in an amount of about 0.1 to about 1.0%, about 0.1%, or about 1.0% by weight.

In certain embodiments, the compositions may be varied to include acids and bases to adjust the pH; tonicity imparting agents such as sorbitol, glycerin and dextrose; other viscosity imparting agents such as sodium carboxymethylcellulose, microcrystalline cellulose, polyvinylpyrrolidone, polyvinyl alcohol and other gums; suitable absorption enhancers, such as surfactants, bile acids; stabilizing agents such as antioxidants, including, without limitation, bisulfites, ascorbates, and D-α-tocopherol (Vitamin E); metal chelating agents, such as sodium edetate; and drug solubility enhancers, such as polyethylene glycols. In certain embodiments, the composition may include an antioxidant in an amount of from about 0.01% to about 1.0%, about 1%, or about 2% by weight.

In certain embodiments, the composition may further include inactive ingredients such as anticoagulants, preservatives, antioxidants and/or any other suitable inactive ingredients known in the art. Such additional ingredients may, for example, be useful to prevent composition degradation over time or facilitate effective use of the composition in physiological systems.

In certain embodiments, the composition may further comprise at least one compound selected from the group consisting of isotonic agents, osmotic pressure controlling agents, serum extending agents and antioxidants.

In certain embodiments, the composition comprises a water-salt medium comprising one or more of sodium salts, potassium salts of chlorides and phosphates. In certain embodiments, the composition further comprises a monosaccharide, preferably mannitol or glycerol, in injection water.

In certain embodiments, the composition may have a concentration of components in the water-salt medium having an osmotic pressure in the range of about 290-600 mosmol/l.

As used herein, D50 (also D(0.5), or d(0.5)), the median, is the particle diameter wherein half of the population of particles lies below this value. Similarly, 90 percent of the particle distribution lies below the D90(D(0.9) or d(0.9)), and 10 percent of the population lies below the D10 (D(0.1) or d(0.1)). Particle sizes may be expressed by weight or volume distribution.

Preferably, the dispersed particles of the emulsion have a monomodal particle size distribution. As used herein, “modality” refers to the number of peaks in the size distribution of particles in the emulsion. A size distribution with one peak is referred to as “monomodal”. A size distribution with more than one peak is referrd to as “multimodal”. The terms “bimodal” and “trimodal” are may be used for size distributions with two or 3 peaks, respectively. Preferably, the compositions are characterized by a monomodal particle size distribution. In certain embodiments, the compositions have a D90 of about 0.260 μm to about 0.300 μm. In certain embodiments, the compositions have a D90 of less than about 0.300 μm, less than about 0.290 μm, less than about 0.280 μm, or less than about 0.270 μm. Preferably, absorption is about 0.1 when particle size is measured using laser diffraction.

Preferably, the compositions are characterized by a particle size distribution of less than about 0.3 μm after sterilization. Sterilization may be by heat sterilization, preferably, autoclaving. In certain embodiments, autoclaving is performed at 121° C. for 15 minutes (1× autoclaving). Autoclaving under these conditions may be repeated, for example, twice (2× autoclaving) or three times (3× autoclaving). In certain embodiments, the compositions are characterized by maintaining a D90 of less than about 0.3 μm after 1× autoclaving. In certain embodiments, the compositions are characterized by maintaining a D90 of less than about 0.4 μm after 2× autoclaving. In certain embodiments, the compositions are characterized by maintaining a D90 of about 0.4 μm or less after 3× autoclaving. In certain embodiments, the compositions maintain a D90 of less than about 0.410 after 3× autoclaving.

In certain embodiments, the composition maintains a D90 of between about 0.2 μm and about 0.4 μm after being autoclaved 1×, 2× or 3×. In certain embodiments, the composition maintains a D90 of between about 0.200 μm and about 0.410 μm after being autoclaved 1×, 2×, or 3×. In certain embodiments, the composition maintains a D90 of between about 0.260 μm and 0.410 μm after being autoclaved 1×, 2×, or 3×.

In certain embodiments, the compositions are characterized by a uniformity of less than about 0.5, less than about 0.4, or less than about 0.3. In certain embodiments, the composition maintains a uniformity of less than about 0.3 after 1× autoclaving. In certain embodiments, the composition maintains a uniformity of less than about 0.3 after 2× autoclaving. In certain embodiments, the composition maintains a uniformity of less than about 0.4 after 3× autoclaving.

Preferably, the compositions are characterized by a serum stability characterized by a particle size distribution of less than about 0.3 μm after about 5 days in serum or ionic solutions. Preferably, the compositions are characterized by a shelf stability of at least about 12 months at 25° C.

In certain embodiments, the composition has a mean particle size equal to or less than about 0.2 μm. In certain embodiments, the composition has a mean particle size in a range of about 0.06 to about 0.2 μm. In certain embodiments, about 95% of the particles have an average particle size of less than about 1.5 μm.

In certain embodiments, the emulsion comprises 90% or more of the total amount by volume of the dispersed particles having a particle size of less than about 0.7 μm. In certain embodiments, the emulsion comprises 50% or more of the total amount by volume of the dispersed particles having a particle size of less than about 0.4 μm.

Another embodiment of the present invention comprises a method for imparting particle size stability to a fluorocarbon emulsion having a discontinuous phase of one or more first fluorocarbons and a continuous aqueous phase, comprising the step of including in the admixture with said first fluorocarbon an emulsion-stabilizing amount of one or more second fluorocarbons having a molecular weight greater than said first fluorocarbon. In certain embodiments, each said second fluorocarbon includes within its structure a lipophilic moiety.

Another embodiment of the invention includes a method for preparing compositions according to the invention, which includes combining an emulsifying agent and a perfluorinated compound to produce a biocompatible and bioinert emulsion. Preferably, the components are emulsified within a continuous aqueous phase. In certain embodiments, the continuous phase of the emulsion may have a pH of about 8.4+/−0.2. Preferably, the components are emulsified at a specific constant pressure. Preferably, the pressure is in the range of about 200 to about 1000 bar.

In certain embodiments, the invention provides a method for producing a perfluorocarbon emulsion, the method comprising: producing a surfactant dispersion in a water-salt medium and homogenization of at least one perfluorocarbon compound in the surfactant dispersion, wherein the resulting composition comprises an emulsion. In certain embodiments, the surfactant dispersion in the water-salt medium is produced by homogenization at a high pressure of at least about 200 to about 1000 bar. Preferably, the surfactant comprises a phospholipid.

In certain embodiments, it is preferable to use about 600 bar pressure and an appropriate number of passes during microfludization to obtain an average droplet size below about 0.2 μm, with a narrow distribution. In certain embodiments, the components are homogenized first to make a primary emulsion, which is then passed through a microfluidizer. In certain embodiments, the microfluidizer has 3 chambers with slit widths of 30, 75, and 400 μm. In certain embodiments, the time for homogenization of the emulsifier and other components before the addition of the PFC may be about 1 minute at between from about 1000 to about 10,000 rpm. In certain embodiments, the homogenization may be at about 8000 rpm. In certain embodiments, it may be preferable to bubble N₂ through the feed and product containers of the high pressure homogenizer to minimize oxidative degradation of surfactant.

Preferably, upon subsequent storage of the emulsion at least about 6 months in a non-frozen state at a temperature of about 25° C., as measured by particle size distribution.

The methods may further comprise heat sterilization of the produced emulsion. In certain embodiments, the composition may be autoclaved for sterilization, preferably at about 121° C. for about 15 min. In certain embodiments, varying ramp up temperature schemes may be used. In certain embodiments, a rotating autoclave may be used to minimize increases in droplet size.

A further embodiment of this invention relates to a formulation comprising a complex comprising oxygen-17 and a composition as described herein. Preferably, the formulation is stable with respect to particle size distribution at room temperature (about 25° C.) for at least about 12 months. Preferably, the formulation is stable with respect to particle size distribution in vivo at human body temperature (about 37° C.) for about 24 hours.

In certain embodiments, a formulation is provided comprising a complex of a composition as described herein and ¹⁷O gas. In certain embodiments, there is provided a formulation comprising a complex of a composition as described herein and ¹⁷O gas, wherein the ¹⁷O gas is at an enrichment of from about 40% to about 90% sauration of the oxygen carrying capacity of the emulsion. Preferably, the formulation comprises oxygen gas at least about 80% saturation of the emulsion.

Oxygen-17 is a commercially available isotope and while not produced in large quantities, can be obtained from several sources. The amount of oxygen-17 actually employed will, of course, depend, in part, on the degree of enhancement of oxygen-17 in the gas. The minimum saturation of Oxygen-17 needed for MRI may vary with the sensitivity of the MRI technical methodology or the pathology being studied. Preferably, saturation of Oxygen-17 gas of about 50% to about 70% may be used in methods and applications described herein. Oxygen-17, which is formed in the manufacture of oxygen-18, is usually obtained in about 70 percent enrichment.

The following provides an exemplary formulation according to an embodiment of the invention:

| Product composition: | % (W/W) |
|---|---|
| Perfluorodecalin | 50% |
| Phospholipon 90G | 5.734 |
| Glycine | 0.636 |
| EDTA disodium | 0.013 |
| Dihydrate | |
| Water for injection | 43.617 |
| NaOH pH adjustment | 8.4 +/− 0.2 |

In certain embodiments, formulations of the invention have the following characteristics:
Particle size distribution 95%<1.5μ (100%<5μ)
Particle distribution of <1 μm: z-average: <=300 nm
Particle distribution of <1 μm: poly-dispersion: <=0.25
sub-visual particle <=100 ml>=10 μm: <=3000/container
sub-visual particle <=100 ml>=25 μm: <=300/container
Oxygen-17 Gas (70%)
99% Pure (in compliance with cGMP 21 Code of Fed Reg. Part 210 and 211)
Emulsion saturated to 99% resulting in Po₂>650 mm of Hg.

Further embodiments of this invention relate to methods of making a formulation comprising a complex of a composition as described herein and oxygen-17 gas. In certain embodiments, the method comprises removing oxygen-16 from the composition prior to loading with oxygen-17 by deoxygenating the composition. In certain embodiments, the composition may be oxygenated by placing a composition comprising an emulsion into an oxygenation loading device and loading the composition into an oxygenator device. In certain embodiments, the oxygenator device comprises a plurality of hollow fiber and/or over the dispersion disc or membranes encased within a larger container, the membranes defining an intracapillary space within the hollow fiber and/or over the dispersion disc and an extracapillary space outside the hollow fiber and/or over the dispersion disc. The method may further include expelling the composition from a oxygenation loading device into a oxygenator device; exposing said composition to oxygen-17 gas by circulating said composition through the intracapillary space within said hollow fiber and/or over the dispersion disc, wherein the oxygen-17 gas remains under positive pressure in the extracapillary space, allowing the composition to draw the oxygen-17 gas across the hollow fiber and/or over the dispersion disc membrane. The oxygen-17 gas may bind with the composition within the hollow fiber and/or over the dispersion disc to form a complex. The complex may be extracted from the hollow fiber and/or over the dispersion disc membrane into a sealed, sterile container. Preferably, the complex remains under positive pressure. In certain embodiments, the oxygenator device includes a sensor that indicates when the complex is formed.

The oxygenator device may comprise a series of hollow fiber and/or over the dispersion disc membrane tubes encased within a larger container. In certain embodiments, the oxygen-17 remains under positive pressure within the larger container while the composition flows through the hollow fiber and/or over the dispersion disc membrane tubes. Once the complex is formed, the formulation remains under positive pressure while the formulation is extracted from the hollow fiber and/or over the dispersion disc membrane into a sealed sterile container.

In certain embodiments, there is provided a method for preparing a formulation comprising:
(a) placing a composition as described herein into an oxygenation loading device;
(b) expelling the composition from the oxygenation loading device into an oxygenator device, wherein the oxygenator device comprises a plurality of hollow fibers and/or at least one over the dispersion disc encased within a larger container, the membranes of the hollow fibers and/or disc defining an intracapillary space within the hollow fibers and/or disc and an extracapillary space outside the hollow fiber and/or disc;
(c) exposing the composition to ¹⁷O gas by circulating the composition through the intracapillary space, wherein the ¹⁷O gas remains under positive pressure in the extracapillary space;
(d) allowing the composition to draw the ¹⁷O gas across the hollow fiber membrane and/or disc;
(e) binding the ¹⁷O gas with the composition within the intracapillary space to form a complex; and
(f) extracting the complex from the intracapillary space into a sealed, sterile container, wherein the complex remains under positive pressure.
   In certain embodiments, the oxygenator device includes a sensor that indicates when the complex is formed.

In a preferred embodiment, the deoxygenated composition, oxygen-17, and the resultant oxygen-17 formulation remain under positive pressure to minimize or completely avoid contamination by oxygen-16. Preferably, there is about 95% saturation of the emulsion maintaining a partial pressure of at least about 650 mm of Hg.

In some instances, it may be desirable to subject the composition to multiple freeze-thaw cycles in order to ensure that removal of all oxygen-16 is complete before introducing the oxygen-17 isotope. Under some circumstances, it might also be desirable to conduct the deoxygenation step under reduced pressure.

In a preferred embodiment, a sealed, sterile container may be selected from a group that includes, but is not limited to, IV bags, syringes, single-use vials, and multiple-use vials.

In certain embodiments, the present invention provides methods involving administration of compositions and/or formulations according to the invention to a subject. As used herein, the term “subject” is used to mean an animal, including, without limitation, a mammal. The mammal may be a human. The terms “subject” and “patient” may be used interchangeably. In certain embodiments, the invention provides for in vivo magnetic resonance imaging of tissue oxygen metabolism in humans.

In certain embodiments, the differentiating and/or monitoring of tissue response to stress may be determined by measuring the rates of production of H₂¹⁷O in a plurality of zones of a tissue of interest in a patient by means of proton magnetic resonance imaging after the patient has been administered an effective amount of a diagnostic imaging agent based on oxygen-17 as described herein. The rates of production between the various zones of a given tissue area in which there is production are compared and the zone(s) in which the rate of production is greater than other zones is identified. Nonviable tissue does not produce water, and this allows viable and nonviable tissue to be distinguished. In certain embodiments, formulations as described herein may be used in a method that looks to the rates of water production in a plurality of zones in the area in which there is production and comparison allows the zones to be distinguished. This may provide information about the effect and effectiveness of therapy to restore viability, tissue regeneration, and the like. The use of proton magnetic resonance imaging after administration of an effective imaging amount of a diagnostic imaging agent comprising a complex of oxygen-17 is described, e.g., in U.S. Pat. No. 4,996,041 and U.S. Pat. No. 7,410,634.

An embodiment of this invention relates to a method of differentiating zones in ischemic tissue by measuring an oxygen extraction fraction in the ischemic tissue by means of a multinuclear (e.g., proton (¹H), oxygen-17 (¹⁷O) or fluorine-19 (¹⁹F)) magnetic resonance imaging system. In certain embodiments, this method may include administering to a subject an effective imaging amount of a formulation described herein, and determining a risk of tissue damage by comparing a first oxygen extraction fraction of a first tissue zone in the ischemic tissue to a second oxygen extraction fraction of a second tissue zone in the ischemic tissue using a magnetic resonance imaging system.

A further embodiment provides a method of differentiating zones of abnormal, reduced blood flow in ischemic tissue by measuring one or more of oxygen delivery, oxygen metabolism and the oxygen extraction fraction in ischemic tissue by means of proton and/or oxygen-17 magnetic resonance imaging, the method comprising:
(a) administering an effective amount of a formulation as described herein to a subject;
(b) measuring one or more of the oxygen delivery, oxygen metabolism and oxygen extraction fraction in tissue with normal blood flow;
(c) measuring the one or more of oxygen delivery, oxygen metabolism and oxygen extraction fraction in one or more zones of tissue with abnormal, reduced blood flow using proton and/or oxygen-17 detection with a magnetic resonance imaging system; and
(d) comparing the measurements obtained in (b) and (c).

The above method may be used to determine the risk of ischemic tissue injury.

In certain embodiments, the invention provides a method of differentiating zones in ischemic tissue by measuring an oxygen extraction fraction in ischemic tissue by means of a proton magnetic resonance imaging system, the method comprising:
(a) administering an effective imaging amount of a formulation of the invention;
(b) measuring a first oxygen extraction fraction of a first tissue zone in the ischemic tissue using the proton magnetic resonance imaging system;
(c) assessing a second oxygen extraction fraction of a second tissue zone in the ischemic tissue using the proton magnetic resonance imaging system; and
(d) determining a risk of tissue damage by comparing the first oxygen extraction fraction of the first tissue zone in the ischemic tissue to the second oxygen extraction fraction of the second tissue zone in the ischemic tissue using the proton magnetic resonance imaging system.

The level of saturation of the formulation to achieve the desired imaging, will depend, in part, on the degree of enrichment of oxygen-17 in the gas. It may also depend on the sensitivity of the MRI technical methodology or the pathology being studied. While an about 99% enrichment may be desired, oxygen-17 is usually supplied in about 70% enrichment. The degree of perfluorocarbon saturation may be appropriately adjusted to optimize MRI sensitivity for the biological research application or clinical pathology being imaged. In certain embodiments, visualization may be achieved with as low as about 80% oxygen saturation of the emulsion. In certain embodiments, the formulation has about 80% to about 99%, about 85% to about 95% or about 95% to about 99% saturation. In some embodiments, the formulation has about 95%, about 96%, about 97%, about 98% or about 99% saturation of the emulsion. Preferably, the formulation maintains a partial pressure of at least about 650 mm of Hg. This provides adequate quantities of oxygen-17 available on the carrier for delivery.

In general, the ratio of oxygen-17 to the composition is dependent on the positive pressure in the loaded emulsion. The ratio of oxygen-17 to the composition is preferably about 1:5 or about 1:7. Thus, in a preferred embodiment, 100 ml of the enriched gas may be complexed with 100 ml of the composition.

Administration of a formulation of the invention as a diagnostic agent may preferably be carried out by intravenous perfusion. A wide variety of methods and instrumentation can be employed to introduce the agent into the body of the subject being examined. Another preferred method is to use a catheter so that the agent can be directed to a desired site in the body and greater control can be obtained of the amount introduced to provide the desired imaging. The catheter also makes it possible to administer therapeutic agents, including, without limitation, thrombolytics, neuoprotective, myoprotective or other agents, after or during the imaging procedure. The formulation employed will be an effective amount necessary to provide the desired imaging and this can vary from a few milliliters to 100 milliliters or more to optimize MRI sensitivity for the biological research application or clinical pathology being imaged. In one embodiment, the effective dosage of the formulation is about 1.0 ml/kg to about 2.5 ml/kg of total body weight.

An advantage of aspects of the present invention is that the formulated imaging agent can be detected using commercially available magnetic resonance equipment with little or no modification. Commercially available MRI units can be characterized by the magnetic field strength used, with a field strength of about 1.5 tesla (T) to 3.0 T as the current typical range used in routine clinical practice and 9.4 T maximum to 0.2 Tesla minimum range currently available for human MRI. For a given field strength, each nucleus has a characteristic frequency which indicates the relative sensitivity of the MRI system to the nucleus, higher frequency equals high sensitivity. For instance, at a field strength of 1.0 Tesla, the resonance (Larmor) frequency for hydrogen is 42.57 MHz; foroxygen-17, 5.694 MHz; for fluorine-19, 39.519; for phosphorus-31, 17.24; and for sodium-23, 11.26 MHz. The frequency ratios between nuclei are fixed so that the hydrogen proton is always the most easily detectable nucleus and the frequencies scale linearly with magnetic field strength (e.g. proton frequency increases to 64 MHz at 1.5 T and 128 MHz at 3.0 T). Higher field strengths improve sensitivity to all nuclei and may be desirable for imaging those nuclei with lower frequencies and sensitivities than hydrogen. Typical clinical magnetic field strengths can be used for the lower sensitivity nuclei by using indirect, proton MRI methods. Proton MRI of oxygen-17 water (¹H₂¹⁷O) is a preferred method for clinical field strength MRI (about 1.5 T to 3.0 T). Moreover, the imaging of different nuclei can be conducted simultaneously or sequentially using combinations of MRI hardware and software.

The methods described herein make possible the non-invasive and visual estimation of the spatial oxygen metabolism distribution in brain and other important organs including, but not limited to, the heart, liver, and kidney, under clinical magnetic resonance systems. Cardiac, visceral, transplant and other tissues also have portions of the areas that may be visualized by MRI which differ from one another in oxygen metabolism. The process of cellular respiration is identical in all tissue and the compensation during metabolic stress is similar albeit the metabolic activity among different tissue types varies based on their function. This means that an ability to differentiate subareas of tissue oxygen metabolism by means of MRI for the evaluation of the reaction to stress may have wide application and is not limited to the evaluation of cerebral tissue.

In certain embodiments, ¹⁷O-MRI may be used to pin-point the seizure focus based on marked elevation of oxygen metabolism during the ictus or reduced inter-ictal oxygen metabolism, enabling physicians to plan surgical resection more accurately.

In certain embodiments, ¹⁷O-MRI can enable physicians to rapidly assess tissue viability and make better in-formed, “personalized” treatment decisions by targeting tissue at highest risk of injury. Unlike gadolinium or iron oxide-based MRI contrast agents, ¹⁷O can cross an intact blood brain barrier to image normal and ischemic cerebral oxygen metabolism (CMRO₂). In addition, an ¹⁷O-MRI can measure myocardial oxygen metabolism (MRO₂).

Different levels of cell injury have corresponding rates of oxygen uptake from the blood (oxygen extraction fraction, OEF) in order to maintain viable levels of oxygen respiratory metabolism: Oxygen-starved ischemic or hypoxic tissue extracts a larger percentage of oxygen than normal tissue while nonviable (intact or necrotic) tissue does not take up any ¹⁷O₂ gas and hence does not produce detectable water (H₂¹⁷O). Conventional MRI used with Oxygen-17 can distinguish hypoxic but viable regions from those in which cell death has occurred due to necrosis and apoptosis.

In certain embodiments, ¹⁷O may be used as a consistent non-invasive biomarker for an investigative compound's mechanism of action at the cellular level and provide a surrogate end point for clinical trials starting from drug discovery thru clinical use. ¹⁷O can also serve as a companion diagnostic to personalize treatment by more specifically targeting treatable tissue

Molecular oxygen levels in neoplastic (cancerous) tissues fluctuate based on the tumor grade and level of oxidative vs. anaerobic metabolism. An ¹⁷O-MRI may be safely track oxygen metabolism changes in tumor tissue before and throughout the course of treatment without exposing the patient to additional radiation.

In further embodiments, other tissue such as, without limitation, lung, bowel and renal are areas in which compounds and methods as described herein can be readily used and the test repeated. This also provides early warning for organ transplant as tissue function can be assessed immediately before, immediately after with drug therapy and its effectiveness can be evaluated over time thereby providing an early warning of transplant rejection.

The visual imaging of the spatial oxygen metabolism distribution in organs gives information about the oxygen delivery to tissues and the utilization of oxygen in such tissue, which is extremely useful to estimate the pathophysiological status of patients in clinical practice.

Potential applications include, without limitation, early detection of tissue viability in cerebral ischemia (stroke), cardiac ischemia (heart attack), muscle ischemia, tumor hypoxia-induced angiogenesis, visualization of tumor hypoxia, tracking tumor response to radiation or chemotherapy, and epilepsy loci mapping.

In certain embodiments, the invention provides a method of differentiating zones within abnormal, reduced blood flow in ischemic tissue by measuring oxygen delivery, oxygen metabolism and/or the oxygen extraction fraction (OEF, which is equivalent to an oxygen extraction ratio, OER) in the ischemic tissue of a subject by means of proton or oxygen-17 magnetic resonance imaging. In certain embodiments, the method comprises (a) administration to a subject of an effective amount of a formulation of the invention, (b) measuring the oxygen delivery, oxygen metabolism and/or oxygen extraction fraction in tissue with normal blood flow and comparing it to that of one or more zones of tissue with abnormal, reduced blood flow using proton detection (preferably T2-weighted or T1p dispersion images of H₂¹⁷O) or direct oxygen-17 detection, or a combination of the two methods (e.g. proton detection with ¹⁷O decoupling) with a magnetic resonance imaging system. In certain embodiments, determination of the risk of ischemic tissue injury may be based on the essential role of vascular delivery of oxygen and oxygen metabolism for survival of all animal and human tissues. Measurement of abnormal oxygen delivery, oxygen metabolism and/or the oxygen extraction fraction may be used as indicators of ischemic tissue injury risk in zones with reduced blood flow in tissues of the body. This assessment of tissue injury risk is of great medical significance in the organs with the highest oxygen metabolism such as the brain (“stroke” risk in cerebral tissue) and heart (“heart attack” risk in cardiac tissue). It is also applicable to other tissues and vital organs including, but not limited to, skeletal muscle, kidney and bowel.

In certain embodiments, methods comprising the measurement of tissue metabolic H₂¹⁷O may include the proton MRI methods of T2-weighted or T1p images of H₂¹⁷O and/or oxygen-17 MRI methods decoupling of the ¹⁷O signal in H₂¹⁷O and direct detection of ¹⁷O signal in H₂¹⁷O using specialized RF transmission and receiver coils.

In certain embodiments, the combined use of formulations as described herein, for example, ¹⁷O-Perfluorodecalin formulations, and MRI measures of blood flow may be employed. The detection of new tissue oxygen-17 water (H₂¹⁷O) with proton or oxygen-17 MRI after the administration of the ¹⁷O formulation is a qualitative indicator of oxygen (¹⁷O₂) delivery and oxidative metabolism (generation of H₂¹⁷O by mitochondrial electron transport and glucose oxidative metabolism). However, semi-quantitative or absolute quantitative determination of the rate of oxygen metabolism and the oxygen extraction fraction (OEF) may require the semi-quantitative or absolute quantitative determination of blood flow to tissue. MRI blood flow methods that may be used include, without limitation: 1) injection H₂¹⁷O for absolute quantitative determination of blood flow, 2) injection of gadolinium (DSC, dynamic susceptibility contrast perfusion) for semi-quantitative determination of blood flow, and 3) arterial spin labeled (ASL) perfusion imaging for absolute quantitative determination of blood flow.

In certain embodiments, methods are provided for the prediction of tissue outcome in cerebral tissue hypoxia and ischemia (stroke). Cerebral tissue has the highest rate of oxygen metabolism in the body and, unlike many other tissues, is almost completely dependent on oxidative metabolism of glucose for energy metabolism. Global or regional hypoxemic or ischemic injury to the brain may be caused by reduced oxygen delivery (e.g. drowning or carbon monoxide breathing) or reduced blood flow (e.g. cardiac arrest or cerebral vascular occlusion, stenosis, vascular spasm or inflammation). The diagnostic use of ¹⁷O formulations as described herein may provide a “bioscale” quantitative measure of impaired oxygen delivery and metabolism, which, combined with assessment of the vascular oxygen extraction fraction (OEF), may provide a means to predicttissue outcome. Aspects of he present invention may be distinguished from methods using ¹⁵O-PET, which is now considered the “gold standard” for quantitative in vivo assessment of tissue and organ oxygen metabolism (Derdeyn C P, Videen T O, Yundt K D, Fritsch S M, Carpenter D A, Grubb R L, Powers W J (2002) Variability of cerebral blood volume and oxygen extraction: stages of cerebral haemodynamic impairment revisited. Brain 125:595-607), by providing a quantitative, noninvasive method for imaging oxygen metabolism that can be simultaneously and directly correlated with conventional MRI methods of tissue viability assessment (for example, diffusion imaging, DWI, perfusion imaging and structural imaging), which are the current “gold standards” for clinical human imaging. The compositions and methods described herein provide images that are more specific to oxygen metabolism because the ¹⁷O₂ gas signal is not confused with the H₂¹⁷O water signal in MRI, in contrast to PET where the radioactive emission from the ¹⁵O₂ gas cannot be distinguished from radioactive emission coming from H₂¹⁵O water. Aspects of the present invention also provide logistical and safety advantages over ¹⁵O-PET by being potentially available on the much larger and growing installed base of clinical MRI scanners compared to PET scanner installations; by obviating the need for expensive radioactive isotope production facilities at the imaging site (T½ of ¹⁵O is 122 seconds and must be produced by a cyclotron at the PET imaging site); and, as a non-radioactive technique, by eliminating the relatively high radiation dose delivered to the body, especially the brain and heart, by ¹⁵O-PET imaging.

These measures of impaired oxygen metabolism are predictive of tissue survival (viability) or injury under hypoxemic (reduced oxygen delivery only, with preservation of blood flow and delivery of other nutrients such as glucose) or ischemic (reduced oxygen delivery and reduced delivery of other nutrients such as glucose because of reduced blood flow) conditions. The potential outcomes of tissue under hypoxemic or ischemic conditions may include survival without injury in regions of mildly reduced oxygen delivery and/or reduced blood flow (“oligemia” with preservation of normal oxygen metabolism and OEF due to a resetting of oxygen demand at a lower level), survival with improved resistance to injury at greater degrees of hypoxemia or ischemia by “preconditioning” in response to the mild hypoxia or ischemia, survival with an increased risk of tissue necrosis or apoptosis in a state of “misery perfusion” with reduced blood flow, preserved of normal or slightly reduced oxygen metabolism but elevated OEF, impending tissue necrosis and irreversible apoptosis with markedly reduced blood flow, reduced oxygen metabolism and elevated OEF, and tissue death from necrosis and apoptosis with reduced blood flow (or belatedly reconstituted blood flow) but absence of oxygen metabolism and OEF. (Heiss, W D, The Ischemic Penumbra: Correlates in Imaging and Implications for Treatment of Ischemic Stroke, Cerebrovasc Dis 2011; 32:307-320). Embodiments include using ¹⁷O formulations for assessment of these states of oxygen metabolism and prediction of tissue survival or injury, as outlined above.

In certain embodiments, methods are provided for prediction of tissue outcome with mechanical injury to brain and/or spinal cord. Cerebral tissue has the highest rate of oxygen metabolism in the body and, unlike many other tissues, is almost completely dependent on oxidative metabolism of glucose for energy metabolism. Global or local mechanical brain/spinal cord injury may be produced by head trauma (TBI, traumatic brain injury), brain hemorrhage or brain mass. The diagnostic use of ¹⁷O formulations as described herein provides a “bioscale” quantitative measure of impaired oxygen delivery and metabolism which, combined with assessment of the vascular oxygen extraction fraction (OEF) provides a means to predict tissue outcome. These measures of impaired oxygen metabolism are predictive of tissue survival or injury produced by diffuse disruption of microvasculature (e.g. DAI, diffuse axonal injury and disruption of arterioles and capillaries with TBI) or local ischemia produced by tissue compression adjacent to hemorrhage or mass lesions. The potential outcomes of tissue under diffuse or local ischemic conditions include survival without injury in regions of mildly reduced blood flow (“oligemia”, with preservation of normal oxygen metabolism and OEF due to a resetting of oxygen demand at a lower level), survival with improved resistance to injury at greater degrees of hypoxemia or ischemia by “preconditioning” in response to the mild hypoxia or ischemia, survival with an increased risk of tissue necrosis or apoptosis in a state of “misery perfusion” with reduced blood flow, preserved of normal or slightly reduced oxygen metabolism but elevated OEF, impending tissue necrosis and irreversible apoptosis with markedly reduced blood flow, reduced oxygen metabolism and elevated OEF, and tissue death from necrosis and apoptosis with reduced blood flow (or belatedly reconstituted blood flow) but absence of oxygen metabolism and OEF. (Signoretti S, Lazzarino G, Tavazzi B, Vagnozzi R., The pathophysiology of concussion. Physical Medicine & Rehabilitation 2011 October; 3(10 Suppl 2):S359-68).

In certain embodiments, methods are provided for the prediction of tissue outcome in the heart and other organs with hypoxia and ischemia. Cardiac and other organ tissues are highly dependent of oxygen for energy metabolism but, unlike brain, may also derive cellular energy from non-oxidative (anaerobic) metabolism of glucose or ketones, for example. The diagnostic use of ¹⁷O formulations as described herein provides a “bioscale” quantitative measure of impaired oxygen delivery and metabolism which, combined with assessment of the vascular oxygen extraction fraction (OEF) still provides a useful means to predict tissue outcome. These measures of impaired oxygen metabolism are predictive of tissue survival or injury under hypoxemic (reduced oxygen delivery only, with preservation of blood flow and delivery of other nutrients such as glucose) or ischemic (reduced oxygen delivery and reduced delivery of other nutrients such as glucose because of reduced blood flow) conditions. The potential outcomes of tissue under hypoxemic or ischemic conditions include survival without injury in regions of mildly reduced oxygen delivery and/or reduced blood flow (“oligemia” or “hibernation” with preservation of normal oxygen metabolism and OEF due to a resetting of oxygen demand at a lower level), survival with improved resistance to injury at greater degrees of hypoxemia or ischemia by “preconditioning” or “hibernating” in response to the mild hypoxia or ischemia, survival with an increased risk of tissue necrosis or apoptosis in a state of “misery perfusion” with reduced blood flow, preserved of normal or slightly reduced oxygen metabolism but elevated OEF, impending tissue necrosis and irreversible apoptosis with markedly reduced blood flow, reduced oxygen metabolism and elevated OEF, and tissue death from necrosis and apoptosis with reduced blood flow (or belatedly reconstituted blood flow) but absence of oxygen metabolism and OEF. (Stanley W C, Recchia F A, Lopaschuk G D. Myocardial substrate metabolism in the normal and failing heart. Physiol Rev 2005; 85:1093-129).

In certain embodiments, methods are provided for the use of an ¹⁷O formulation as a “companion diagnostic” agent to target and monitor therapy for hypoxia and ischemia. As used herein, “companion diagnostic” refers to a diagnostic agent that may be used to guide therapy. For example, this embodiment of the invention can be combined with specific therapies for reconstitution or improvement of blood flow to ischemic tissue, such as IV or IA thrombolysis, anticoagulation, plate inhibition, rheological agents and elevation of systemic blood pressure. This embodiment of the invention can be used to improve the specificity and effectiveness of pharmacologic therapies as well as “physiologic” therapies such as hyperbaric or normobaric 100% oxygen breathing for hypoxic/ischemic tissue injury. Another potential application is the use of ¹⁷O formulations to target early or minimal stages of oxygen metabolism changes that produce “oxidative stress” which triggers apoptotic cell death, thus providing a target with high likelihood of success for interruption of the early apoptotic enzymatic cascade (e.g. cerebral “neuroprotective” treatment strategies). (Nakka, V. P.; Gusain, A.; Mehta, S. L.; et al., Molecular mechanisms of apoptosis in cerebral ischemia: Multiple neuroprotective opportunities, Molecular Neurobiology (2008) 37: 7-38).

In a further embodiment, methods are provided for the combined use of proton MRI, oxygen-17 MRI and fluorine-19 (¹⁹F) MRI for monitoring ¹⁷O₂ oxygen delivery, oxygen metabolism and/or the oxygen extraction fraction as well as tissue levels of ¹⁶O₂. In addition to the use of proton and oxygen-17 MRI as described above, direct detection of the stable fluorine-19 in the perfluorocarbon nanomolecular oxygen carrier component of the invention can be performed with the same MRI system. This can be done with established technology using proton detection coils (¹⁹F has a high gyromagnetic ratio, similar to ¹H protons) or specialized detection coils specifically tuned to the magnetic resonance frequency of the ¹⁹F nucleus. (Kaneda M M, Caruthers S, Lanza G M, Wickline S A. Perfluorocarbon nanoemulsions for quantitativemolecular imaging and targeted therapeutics. Ann Biomed Eng 2009, 37:1922-1933).

Quantitative images of the distribution of the perfluorocarbon agent can be produced with high accuracy as there is no background ¹⁹F signal in the human body soft tissues (The only ¹⁹F is in teeth and bones which is MRI “invisible” as it is in a solid state and does not produce detectable MRI signal). These ¹⁹F MR images can provide a quantitative, regional, tissue level assessment of the concentration of the perfluorocarbon ¹⁷O₂ carrier for improved quantitation of local ¹⁷O₂ delivery (with consequent improved accuracy of local oxygen metabolism and OEF determinations). The quantitative assessment of ¹⁷O₂ delivery can be calculated from the known concentration of ¹⁷O₂ on the perfurocarbon carrier when injected intravenously or intra-arterially. It can also be calculated by changes in the fluorine MRI signal caused by changes in the relaxation properties of ¹⁹F which are known to be directly sensitive to the local concentration of oxygen. (Kodibagkar V D, Wang X, Mason R P. Physical principles of quantitative nuclear magnetic resonance oximetry. Front Biosci 2008, 13:1371-1384).

The oxygen sensitivity of the ¹⁹F signal, therefore, can also be used to assess the local concentration of ¹⁶O₂ delivered to the tissue by the perfluorocarbon carrier after it recirculates through the lungs and becomes saturated with room air or hyperbaric or normobaric 100% oxygen.

In additional embodiments, methods are provided for the combined use of an ¹⁷O formulation, proton MRI, oxygen-17 MRI and fluorine-19 MRI as a “companion diagnostic” agent to target and monitor therapy for neoplastic tissue. The oxygen content of neoplastic tissue can be calculated by changes in the fluorine MRI signal caused by changes in the relaxation properties of ¹⁹F which are known to be directly sensitive to the local concentration of oxygen. The combined use of ¹⁷O formulations to identify suspected tumor tissue with low oxygen metabolism (and low OEF) in the presence of normal or high oxygen levels identified by ¹⁹F MRI can act as an indirect indicator of a preferential shift toward “anaerobic glycolysis” in the presence of adequate oxygen (the Warburg effect) that is characteristic of aggressive cancerous tissue. Normal OEF in hypoxic tumor is also an indicator of preferential anaerobic glycolysis (i.e. no elevated OEF in the presence of reduced oxygen delivery indicates a metabolic “preference” for anaeribic glycolysis, which is the Warburg effect). (Melillo G. Targeting hypoxia cell signaling for cancer therapy. Cancer Metastasis Rev 2007, 26:341-352).

This approach may provide a method of “grading” neoplastic tissue on the basis of its metabolic state and provide a “companion diagnostic” agent to help target cancer therapies (e.g. chemotherapy, immunotherapy, etc.) or to monitor treatment response or failure. It may also provide a method to identify high local concentrations of ¹⁶O₂ which may act as a guide for radiation therapy; radiation produces radical oxygen species (ROS), or “free radicals”, that are the main mechanism of cell death produced by radiation therapy.

Additional embodiments provide methods for the combined use of ¹⁷O formulations, proton MRI and oxygen-17 MRI for ¹⁷O with the use of proton MRI for ¹⁶O₂ detection as a “companion diagnostic” agent to target and monitor therapy for neoplastic tissue. One embodiment relates to the combined use of ¹⁷O formulations as described herein to identify suspected tumor tissue with low oxygen metabolism (and low OEF) in the presence of normal or high oxygen levels identified by a proton MRI method such as T1 relaxivity (R1) (L. E. Kershaw, J. H. Naish, D. M. McGrath, J. C. Waterton, G. J. M. Parker. (2010). Measurement of arterial plasma oxygenation in dynamic oxygen-enhanced MRI. Magnetic Resonance in Medicine, 64, 1838-1842) or blood oxygen dependent (BOLD) susceptibility weighted (EM Haacke, J Tang, J Neelavalli, Y C N Cheng, Susceptibility Mapping as a Means to Visualize Veins and Quantify Oxygen Saturation J Magn Reson Imaging. 2010 September; 32(3): 663-676; Yablonskiy D A, Haacke E M. Theory of NMR signal behavior in magnetically inhomogeneous tissues: the static dephasing regime. Magn Reson Med. 1994; 32:749-763).

MRI can act as an indirect indicator of a preferential shift toward “anaerobic glycolysis” in the presence of adequate oxygen (the Warburg effect) that is characteristic of aggressive cancerous tissue. Normal OEF in hypoxic tumor is also an indicator of preferential anaerobic glycolysis (i.e. no elevated OEF in the presence of reduced oxygen delivery indicates a metabolic “preference” for anaeribic glycolysis, which is the Warburg effect). This approach may provide a method of “grading” neoplastic tissue on the basis of its metabolic state and provide a “companion diagnostic” agent to help target cancer therapies (e.g. chemotherapy, immunotherapy, etc.) or monitor treatment response or failure. It may also provide a method to identify high local concentrations of ¹⁶O₂ which may act as a guide for radiation therapy (radiation produces radical oxygen species (ROS), or “free radicals”, that are the main mechanism of cell death produced by radiation therapy).

Further embodiments provide methods of combined therapeutic and diagnostic or “theranostic” application of ¹⁷O formulations as described herein. ¹⁷O formulations may be used to diagnose the degree of hypoxia or ischemia in cerebral, cardiac or other tissue during the “first pass” delivery of the ¹⁷O₂ by the perfluorocarbon carrier, as described above. This is followed by recirculation of the perfluorocarbonthrough the lungs where it is enriched with room air or hyperbaric/normaric high ¹⁶O₂ concentration that is subsequently delivered to the tissue, a therapeutic application of the invention. The small particle size of the perfluorcarbon is key to its therapeutic role, as it improves oxygen delivery to tissue by two mechanisms: 1) facilitated diffusion through blood plasma from the hemoglobin in RBC's to the tissue and 2) delivery of oxygen to tissues that are not accessible to RBC's or free hemoglobin (e.g. through partially thrombosed vessels or partially collapsed capillaries). (Speiss B D, Perfluorocarbon emulsions as a promising technology: a review of tissue and vascular gas dynamics, J Appl Physiol 106:1444-1452, 2009).

In certain embodiments, methods are provided comprising the formation of perfluorocarbon microbubbles filled with ¹⁷O₂ gas using established methods for the production of medical ultrasound contrast agents. In certain embodiments, methods comprise the use of a medical ultrasound probe to disrupt these microbubbles in the vascular supply to the tissue of interest (e.g. the carotid artery for brain tissue). This process may provide a more targeted delivery of ¹⁷O₂ gas at high concentration that the use of passive adsorption of the ¹⁷O₂ gas on the perfluorocarbon carrier. (S. R. Sirsi and M. A. Borden, Microbubble compositions, properties and biomedical applications, Bubble Science, Engineering and Technology 2009 1:1-17).

In certain embodiments, the imaging agent formulation and the method of use as described herein, may be characterized by several other desirable features. Since all of the oxygen-17 employed can be complexed with the composition prior to use, complete control can be maintained over the amount of the isotope used and little, if any is lost as would be the case if administered by inhalation. Moreover, the diagnostic agent according to aspects of this invention is easily produced and the resulting formulation may be administered intravenously in the same manner as a venous transfusion. Moreover, when used in conjunction with a catheter, the formulation may be delivered directly to the tissue under study.

A further embodiment of this invention relates to cerebral tissue-specific targeting via passage through the blood brain barrier using a perfluorocarbon emulsion comprising a polysorbate surfactant. Multiple different nanoparticles coated with polysorbate 20, 40, 60 or 80 are known to be absorbed through the blood brain barrier and have been used to facilitate drug delivery to cerebral tissue. While not intending to be bound by any theory of operation, the mechanism for this blood brain barrier penetration appears to be the adsorption of apolipoproteins by polysorbates from the blood which allows them to mimic lipoproteins and induce endothelial cell receptor mediated endycytosis. Drugs transported through the blood brain barrier by this mechanism may then freely diffuse within the cerebral cellular matrix or be incorporated into cerebral cells via transcytosis. (Krueter, et. al., Apolipoprotein-mediated Transport of Nanoparticle-bound Drugs Across the Blood-Brain Barrier Journal of Drug Targeting, 2002 Vol. 10 (4), pp. 317-325). This mechanism may be active in the delivery of ¹⁷O₂ to cerebral tissue in addition to simple oxygen diffusion. In certain embodiments, this mechanism for facilitating passage through the blood brain barrier may be applied to other agents or drugs which are soluble in the perfluorocarbon emulsions described herein for the purpose of targeted delivery to cerebral tissue.

A further embodiment of this invention relates to kits including sterile containers containing a formulation disclosed herein, while, preferably, the formulation remains under positive pressure. Preferably, the container is sealed and sterile. Preferably, the container may be selected from a group consisting of, but not limited to, IV bags, syringes, single-use vials, and multiple-use vials.

It will be apparent to those skilled in the art that various modifications and variations can be made in the methods and compositions of the present inventions without departing from the spirit or scope of the invention. Thus, it is intended that the present invention cover the modification and variations of the inventions provided they come within the scope of the appended claims and their equivalents.

In addition, where features or aspects of the invention are described in terms of Markush group or other grouping of alternatives, those skilled in the art will recognized that the invention is also thereby described in terms of any individual member or subgroup of members of the Markush group or other group.

Unless indicated to the contrary, all numerical ranges described herein include all combinations and subcombinations of ranges and specific integers encompassed therein. Such ranges are also within the scope of the described invention.

All references cited herein are incorporated by reference herein in their entireties.

The following examples serve to further illustrate the present invention.

### Example 1

The emulsions of Examples 1-3 comprising perfluorodecalin were prepared using procedures described in U.S. Patent Application No. 2010/0267842.

### Emulsion 1

| Component | Purpose | % (w/w) |
|---|---|---|
| Perfluorodecalin | Oxygen carrier | 50.00 |
| Soybean oil | Stabilizer | 2.00 |
| Glycine | Buffer | 0.64 |
| Lipoid E80 | Surfactant | 4.04 |
| EDTA disodium dihydrate | Trace Metal | 0.10 |
| | scavenger | |
| D-α-tocopherol(Vitamin E) | Antioxidant | 1.0 |
| Glycerol or Sodium Chloride | Adjust | As needed |
| | emulsion | |
| | osmolarity | |
| Water for injection | Continuous | 42.22 |
| | Phase | |
| Base (NaOH or NaHCO₃) for pH | Maintain a pH | As needed |
| adjustment | of 8.4 | |

### Example 2

### Emulsion 2

| Component | Purpose | % (w/w) |
|---|---|---|
| Perfluorodecalin | Oxygen carrier | 50.00 |
| Glycine | Buffer | 0.64 |
| Lipoid E80 | Surfactant | 5.73 |
| EDTA disodium dihydrate | Trace Metal | 0.01 |
| | scavenger | |
| D-α-tocopherol(Vitamin E) | Antioxidant | 1.0 |
| Glycerol or Sodium Chloride | Adjust | As needed |
| | emulsion | |
| | osmolarity | |
| Water for injection | Continuous | 42.62 |
| | Phase | |
| Base (NaOH or NaHCO₃) for pH | Maintain a pH | As needed |
| adjustment | of 8.4 | |

### Example 3

### Emulsion 3

| Component | Purpose | % (w/w) |
|---|---|---|
| Perfluorodecalin | Oxygen carrier | 50.00 |
| Glycine | Buffer | 0.64 |
| Lipoid E80 | Surfactant | 5.73 |
| EDTA disodium dihydrate | Trace Metal | 0.01 |
| | scavenger | |
| Glycerol or Sodium Chloride | Adjust | As needed |
| | emulsion | |
| | osmolarity | |
| Water for injection | Continuous | 43.62 |
| | Phase | |
| Base (NaOH or NaHCO₃) for pH | Maintain a pH | As needed |
| adjustment | of 8.4 | |

The resulting perfluorodecalin emulsions of Examples 1-3 are stable with respect to particle size for 12 months at 25° C. and have a D(0.9) value of about 0.3 μm; and a D(0.5) value of about 0.15 μm.

### Example 4

The emulsions of Examples 4-9 were prepared using procedures described in U.S. Patent Application No. 2010/0267842. The particle size distributions are expressed as volume distributions. Sterilization was performed by autoclaving at 121° C. for 15 minutes.

| Component | % w/w |
|---|---|
| Perfluorodecalin | 50.00 |
| Perfluorodecyl bromide | 10.00 |
| Soybean oil | 0.00 |
| Glycine | 0.64 |
| EDTA disodium dihydrate | 0.10 |
| Water for Injection | 33.22 |
| S75 | 0.00 |
| E80 | 4.04 |
| Phosphatidic acid | 0.00 |
| D-α-tocopherol (Vitamin E) | 2.00 |
| NaCl | As needed |
| NaOH pH adjustment | pH = 8.4 ± 0.2 |

The above emulsion of Example 4 was not homogenous.

### Example 5

| Component | % w/w |
|---|---|
| Perfluorodecalin | 50.00 |
| Perfluorodecyl bromide | 0.00 |
| Soybean oil | 10.00 |
| Glycine | 0.64 |
| EDTA disodium dihydrate | 0.10 |
| Water for Injection | 33.22 |
| S75 | 0.00 |
| E80 | 4.04 |
| Phosphatidic acid | 0.00 |
| D-α-tocopherol (Vitamin E) | 2.00 |
| NaCl | As needed |
| NaOH pH adjustment | pH = 8.4 ± 0.2 |

The above emulsion of Example 5 demonstrated good particle size distribution after homogenization, with a D(0.9) value of 0.294 μm, and a D(0.5) value of 0.148 μm, and a D(0.1) value of 0.071 μm. Uniformity was 0.467. After 1× sterilization, however, the particle size distribution was bimodal. The D(0.9) value was 9.904 μm, the D(0.5) value was 5.964 μm, and the D(0.1) was 0.694 μm. Uniformity was 0.391.

### Example 6

| Component | % w/w |
|---|---|
| Perfluorodecalin | 50.00 |
| Perfluorodecyl bromide | 0.00 |
| Soybean oil | 0.00 |
| Glycine | 0.64 |
| EDTA disodium dihydrate | 0.10 |
| Water for Injection | 43.62 |
| S75 | 5.73 |
| E80 | 0.00 |
| Phosphatidic acid | 0.00 |
| D-α-tocopherol (Vitamin E) | 0.00 |
| NaCl | As needed |
| NaOH pH adjustment | pH = 8.4 ± 0.2 |

The above emulsion of Example 6 demonstrated good particle distribution after homogenization (before sterilization): D(0.9) value of 0.2 04 μm, D(0.5) value of 0.117, and D(0.1) value of 0.069 μm. Uniformity was 0.356. After sterilization, larger particles were formed. After 1× sterilization, it had a (D(0.9) value of 0.390 μm a D(0.5) value of 0.183 μm, and a D(0.1) of 0.084 μm. Uniformity of 1.93. After 2× sterilization, the D(0.9) value was 9.866 μm, the D(0.5) value was 0.311 μm, and D(0.1) was 0.120. Uniformity was 15.9. After 3× sterilization, the D(0.9) value was 4.883 μm, the D(0.5) value was 0.289 μm, and D(0.1) was 0.105 μm. Uniformity was 6.47.

### Example 7

| Component | % w/w |
|---|---|
| Perfluorodecalin | 50.00 |
| Perfluorodecyl bromide | 0.00 |
| Soybean oil | 0.00 |
| Glycine | 0.64 |
| EDTA disodium dihydrate | 0.01 |
| Water for Injection | 43.62 |
| S75 | 0.00 |
| E80 | 5.73 |
| Phosphatidic acid | 0.00 |
| D-α-tocopherol (Vitamin E) | 0.00 |
| NaCl | As needed |
| NaOH pH adjustment | pH = 8.4 ± 0.2 |

The above emulsion of Example 7 demonstrated good particle distribution after homogenization and before sterilization, with a D(0.9) value of 0.176 μm, a D(0.5) value of 0.110 μm, and a D(0.1) value of 0.071 μm. The uniformity was 0.299. The emulsion showed a particle distribution after 1× sterilization having a D(0.9) value of 0.270 μm, a D(0.5) value of 0.133 μm, and a D(0.1) value of 0.066 μm. The uniformity was 0.473. After 2× sterilization, the emulsion demonstrated a D(0.9) value of 0.369 μm, a D(0.5) value of 0.154 μm, and a D(0.1) value of 0.071. Uniformity was 0.639. After 3× sterilization, the emulsion had a D(0.9) value of 0.710 μm, a D(0.5) value of 0.180 μm and a D(0.1) of 0.075 μm. The uniformity was 20.3.

### Example 8

| Component | % w/w |
|---|---|
| Perfluorodecalin | 50.00 |
| Perfluorodecyl bromide | 10.00 |
| Soybean oil | 0.00 |
| Glycine | 0.64 |
| EDTA disodium dihydrate | 0.10 |
| Water for Injection | 32.22 |
| S75 | 0.00 |
| E80 | 4.04 |
| Phosphatidic acid | 1.00 |
| D-α-tocopherol (Vitamin E) | 2.00 |
| NaCl | As needed |
| NaOH pH adjustment | pH = 8.4 ± 0.2 |

The emulsion of Example 8 above was not homogenous.

### Example 9

| Component | % w/w |
|---|---|
| Perfluorodecalin | 50.00 |
| Perfluorodecyl bromide | 0.00 |
| Soybean oil | 10.00 |
| Glycine | 0.64 |
| EDTA disodium dihydrate | 0.10 |
| Water for Injection | 32.22 |
| S75 | 0.00 |
| E80 | 4.04 |
| Phosphatidic acid | 1.00 |
| D-α-tocopherol (Vitamin E) | 2.00 |
| NaCl | As needed |
| NaOH pH adjustment | pH = 8.4 ± 0.2 |

The emulsion of Example 9 above had a D(0.9) value of 0.203 μm, a D(0.5) value of 0.121 μm and a D(0.1) value of 0.072 μm after homogenation, but before sterilization. Uniformity was 0.336. After sterilization, measurements were not obtained due to the high viscosity of the samples.

### Example 10

### O₂ Adsorption in a PFC Emulsion Under Pressure

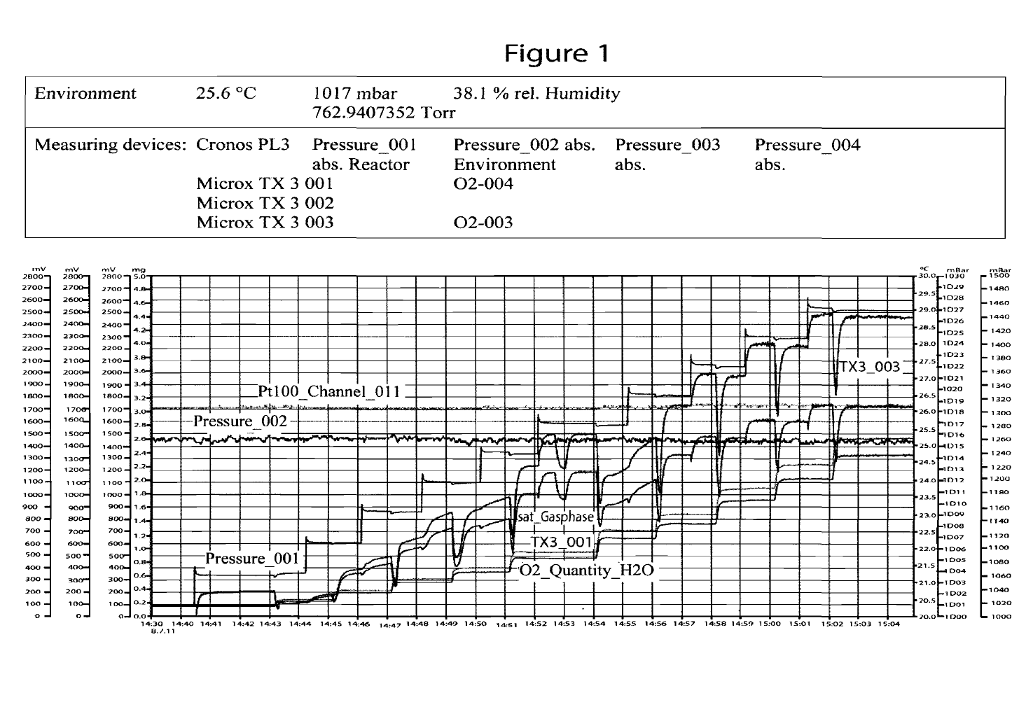

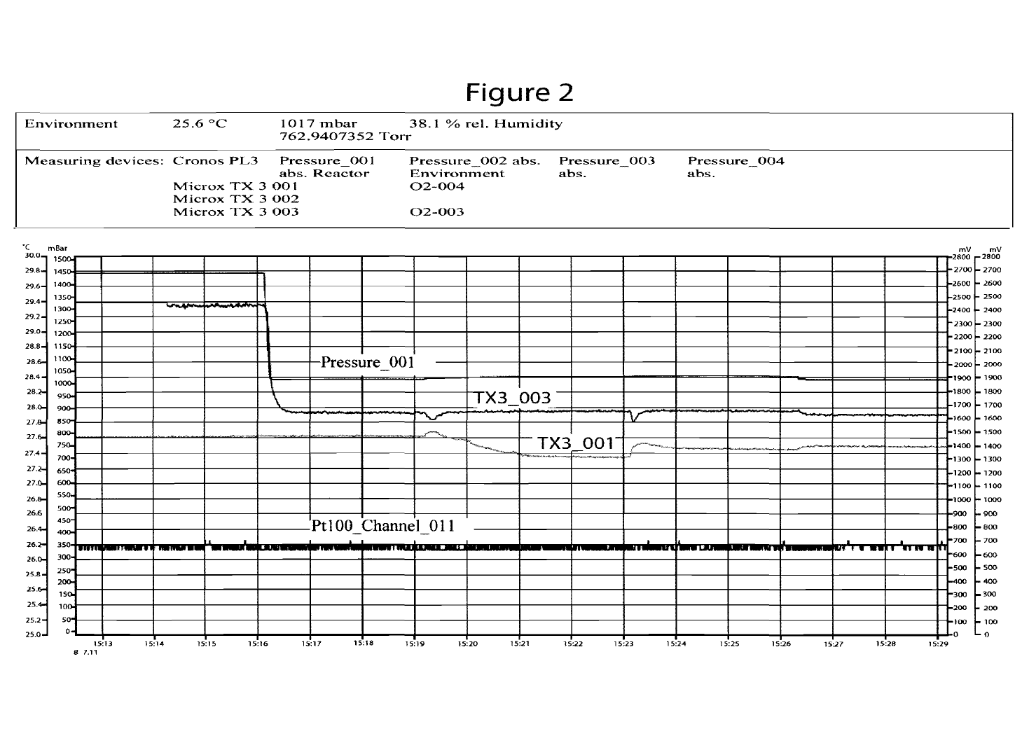

The O₂ uptake of perfluorcarbon (PFC) emulsions under pressure was tested using the perfluorodecalin emulsions of Examples 1, 2, and 3 with distilled H₂O as a control liquid. The oxygen-carrying capacity of the emulsions is dependent upon the concentration of perfluorodecalin, which was 50% for each of the emulsions tested. FIGS. 1 and 2 show representative results for such emulsions.

For the results shown in FIG. 1, the following conditions were used:

Channel

TX3_{—}001: 1100 mV=110% air saturation in the water phase

TX3_{—}003: 1100 mV=110% air saturation in the gas phase

Values are not temperature compensated.

| | |
|---|---|
| 13:41:00 | 200 ml distilled H₂O were filled under N₂ into a 500 ml bottle. |
| | Application of 10 times 20 ml of O2 (normal pressure) into the bottle. |
| | During experiment is the O₂ content is measured. |
| | Liquid phase analog output TX3_001 “Oxygen air saturation” |
| | Gas phase analog output TX3_003 “Oxygen air saturation” |
| 14:39:00 | Start measuring |
| | Gas phase normal pressure in N₂ |
| 14:40:00 | Gas phase loaded with 20 ml O₂ |
| 14:43:00 | Shaking of bottle |
| 14:44:00 | Gas phase loaded with 20 ml O₂ |
| 14:45:00 | Shaking of bottle |
| 14:46:00 | Gas phase loaded with 20 ml O₂ |
| 14:47:00 | Shaking of bottle |
| 14:48:00 | Gas phase loaded with 20 ml O₂ |
| 14:49:00 | Shaking of bottle |
| 14:50:00 | Gas phase loaded with 20 ml O₂ |
| 14:51:00 | Shaking of bottle |
| 14:52:00 | Gas phase loaded with 20 ml O₂ |
| 14:53:00 | water drop at sensor head |
| 14:54:00 | Shaking of bottle |
| 14:55:00 | Gas phase loaded with 20 ml O₂ |
| 14:56:00 | Shaking of bottle |
| 14:57:00 | Gas phase loaded with 20 ml O₂ |
| 14:58:00 | Shaking of bottle |
| 14:59:00 | Gas phase loaded with 20 ml O₂ |
| 15:00:00 | Shaking of bottle |
| 15:01:00 | Gas phase loaded with 20 ml O₂ |
| 15:02:00 | Shaking of bottle |
| 15:04:00 | END |

As demonstrated in FIG. 1, significant loading of perfluorcarbon (PFC) with O₂ can be achieved by simple shaking or stirring of the emulsion within the gas phase. When O₂ was applied under pressure in a shaking reactor vessel, the existence of micro bubbles and the connected measuring errors of O₂ concentrations were negligible.

After shaking, all visible bubbles quickly move from the liquid phase to the surface of emulsion, were no measurement takes place. Measurement variations due to micro bubbles would be recognized by the sensor, because O₂ bubbles are collected at the sensor head. However, while moving the sensor through the emulsion no variation of measuring results could be recognized.

By constantly stirring of the emulsion after pressure decrease a blistering can be prevented as well. The gas exchange between liquid phase and gas phase by stirring is sufficiently fast. The concentration of O₂ in the liquid phase is kept constant by stirring the emulsion. Local differences in concentration are not sufficient to form bubbles. A similar behavior may be recognized in O₂ transport in blood.

The performed tests show that the PFC emulsion can be easily loaded by simple measures. While not intending to be bound by any theory of operation, the above O₂ measurement technique may only partially detect the O₂ in the PFC emulsion, and therefore represents qualitative results.

The pressure increase shows clearly the application of O₂. While shaking the PFC emulsion the pressure decrease can be better recognized than in the H₂O trials. The pressure drop increases with increasing total pressure from about 1 mbar to more than 3 mbar.

The pressure compensated saturation in the gas phase reaches 173% at the end. (184% in H₂O trial). The pressure in the reactor is 1449 mbar at the end. (1474 mbar in H₂O trial). The O₂ amount in the water phase is 2.37 mg at a saturation of 146% air saturation. (227% in H₂O trial)

### Example 11

### O₂ Release in a Loaded PFC Emulsion after Rapid Pressure Drop

Additional testing was performed to determine how the O₂ concentration changes if the pressure drops.

For the results shown in FIG. 2, the following conditions were used:

### Channel

TX3_{—}001: 1100 mV=110% air saturation in the water phase
TX3_{—}003: 1100 mV=110% air saturation in the gas phase
Values are not temperature compensated

### Time

| | |
|---|---|
| 15:11:00 | 200 ml PFC 01.02 loaded with O₂ from the previous trial under |
| | pressure at 1446 mbar. |
| | Rapid pressure release. |
| | Liquid phase analog output TX3_001 “Oxygen air saturation” |
| | Gas phase analog output TX3_003 “Oxygen air saturation” |
| 15:14:00 | Start measuring |
| 15:16:00 | Valve open |
| 15:18:00 | Valve closed |
| 15:19:00 | Shaking of bottle |
| 15:23:00 | Shaking of bottle |
| 15:26:00 | Pressure release |
| 15:29:00 | 2x TX3 stop |
| 15:29:00 | END |

As demonstrated in FIG. 2, micro bubbles and formation of bubbles after a sudden decrease of pressure in the reactor vessel is observed only after a considerable period of time (about 4 hours without stirring the liquid) and only at condensation points. And this is only observed if the emulsion takes on much more O₂ due to excess pressure than it would adsorb under normal conditions.

In FIG. 2, the saturation of the gas phase before opening the valve is 244% air saturation. While opening the valve, the N₂/O₂ gas mixture escapes. The pressure drop leads to a reduction of saturation to about 164%. The saturation in the water phase remains stable, since neither the current temperature changes, nor is the partial pressure in water adjusted rapidly to the environment. Only by shaking the reactor at 15:19:00 and 15:23:00 was change observed.

During the measurement, a clear relation between saturation in the gas phase and in the liquid phase can be recognized. The shaking of the reactor two times leads to an exchange of concentration between the two phases. After the first shaking at the expense of the liquid phase, air saturation goes from 147% to 131%. After the second shaking in favor of the liquid phase, it goes from 131% to 137%.

A significant reduction in O₂ concentration from 249% to 186%, as in the experiment with H₂O with nearly 25% reduction, does not appear.

### Example 12

To determine the effect of the egg phospholipid (Lipoid) content upon ripening, additional emulsion compositions (described in this Example and Example 13) were prepared. The following composition was prepared as follows. Lipoid, polysorbate 20 (Tween® 20), glycerin, EDTA, soybean oil and water were weighed in a beaker, warmed slightly and homogenized. Perfluordecalin was weighed and homogenized. The composition was passed through a M-110P microfluidizer (Microfluidics) at 27,000 psi for 5 passes. Particle sizes were analyzed by laser diffraction using a Mastersizer 2000 (Malvern). The composition was autoclaved one time at 121° C. for 15 minutes, then centrifuged for 5 min and the particle size distribution (PSD) analysis was run. The particle size distributions are expressed as volume distributions. After autoclaving and centrifugation, the PSD values were as follows: D10 was 135 nm, D50 was 186 nm, and D90 was 270 nm.

| Component | % w/w |
|---|---|
| Perfluorodecalin (cGMP) | 50 |
| Lipoid E80 | 5 |
| Tween 20 | 2 |
| Sodium EDTA | 0.01 |
| Glycerin | 3 |
| Soybean oil | 1 |
| Water | 39 |

### Example 13

The composition below was prepared and analyzed according to the method described in Example 12. This composition and that shown in Example 12 showed similar PSD values before autoclaving.

| Component | % w/w |
|---|---|
| Perfluorodecalin (cGMP) | 50 |
| Lipoid E80 | 4 |
| Tween 20 | 2 |
| Sodium EDTA | 0.01 |
| Glycerin | 3 |
| Soybean oil | 1 |
| Water | 40 |

After autoclaving and centrifugation, the D10 was 135 nm, the D50 was 186 nm, and the D90 was 270 nm. The particle size distributions are expressed as volume distributions. The results of this Example and of Example 12 demonstrate that the stability of the particle size distribution (as measured after autoclaving) is enhanced by the addition of polysorbate 20.

### Example 14

Examples of the following composition were prepared according to the method described in Example 16. Glycerol was adjusted as needed to maintain osmolarity between 300-450 mosmols. Particle sizes were analyzed by laser diffraction using a Mastersizer 2000 (Malvern). The particle size distributions are expressed as volume distributions.

| Component | % w/w |
|---|---|
| Perfluorodecalin (cGMP) | 50 |
| Lipoid E80 | 5 |
| Tween 20 | 2 |
| Sodium EDTA | 0.01 |
| Glycerol—Adjust as needed | 3 |
| Soybean oil | 2 |
| Glycine | 0.06 |
| Water for Injection | 37.93 |
| pH adjustment with NaOH or HCl, 0.01N | pH to 8.4 |

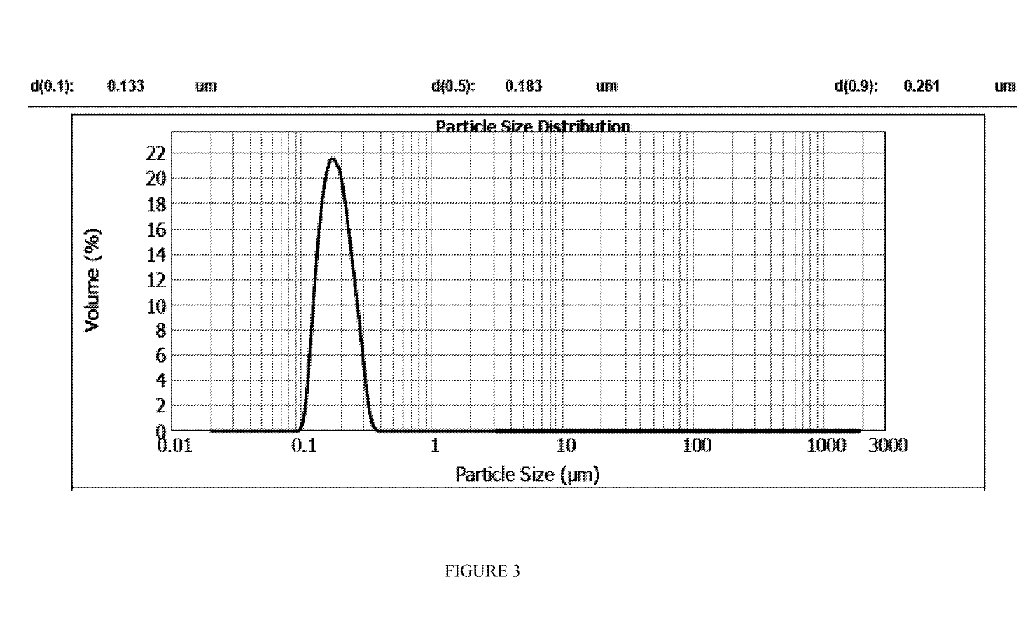

After 5 passes through the microfluidizer at 27,000 psi, but without autoclaving, the composition had the following particle size distribution: d(0.1) was 0.133 μm, d(0.5) was 0.183 μm, and d(0.9) was 0.261 μm. Uniformity was 0.213. Surface weighted mean (D[3,2]) was 0.179 μm. Volume weighted mean (D[4,3]) was 0.191 μm. Absorption was 0.1. The particle size distribution is shown in FIG. 3.

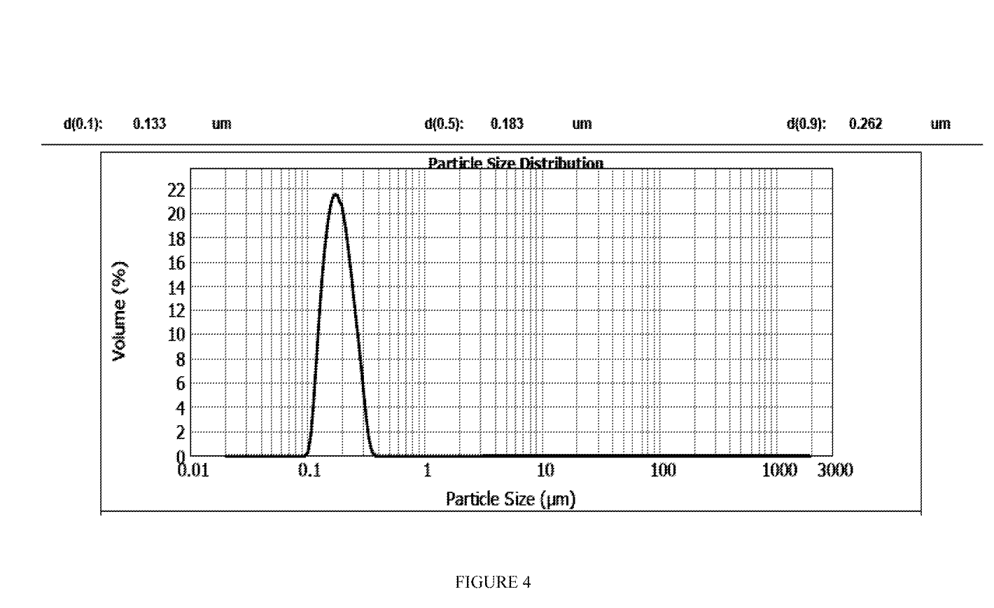

After being autoclaved 1× at 121° C. for 15 minutes, such a composition had a d(0.1) of 0.133 μm, a d(0.5) of 0.183 μm, and a d(0.9) of 0.262 μm. Uniformity was 0.214. D[3,2] was 0.179 μm. D[4,3] was 0.191 μm. Absorption was 0.1. The particle size distribution is shown in FIG. 4.

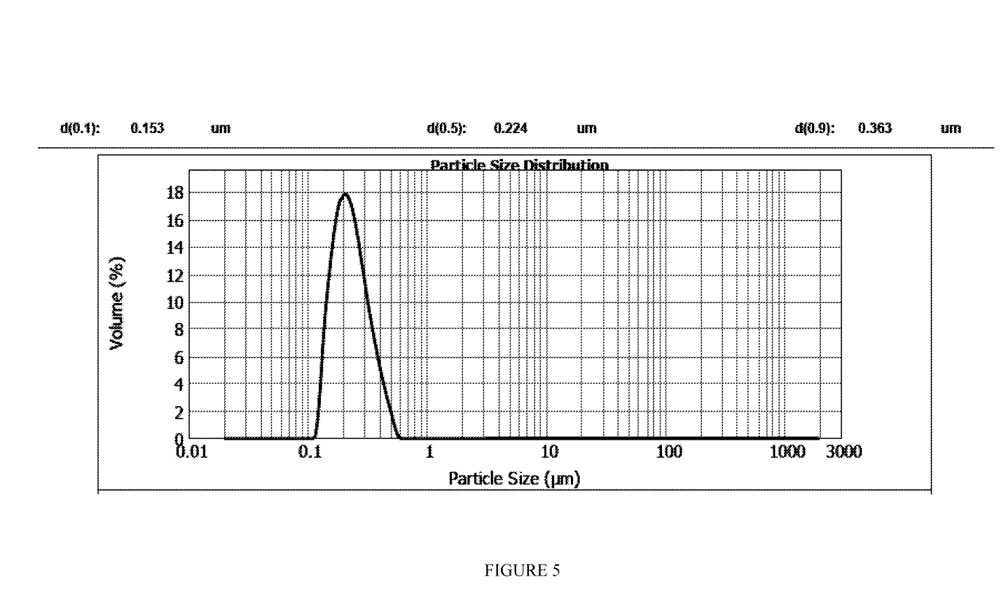

After being autoclaved 2× at 121° C. for 15 minutes, such a composition had a d(0.1) of 0.153 μm, a d(0.5) of 0.224 μm, and a d(0.9) of 0.363 μm. Uniformity was 0.29. D[3,2] was 0.219 μm. D[4,3] was 0.243 μm. Absorption was 0.1. The particle size distribution is shown in FIG. 5.

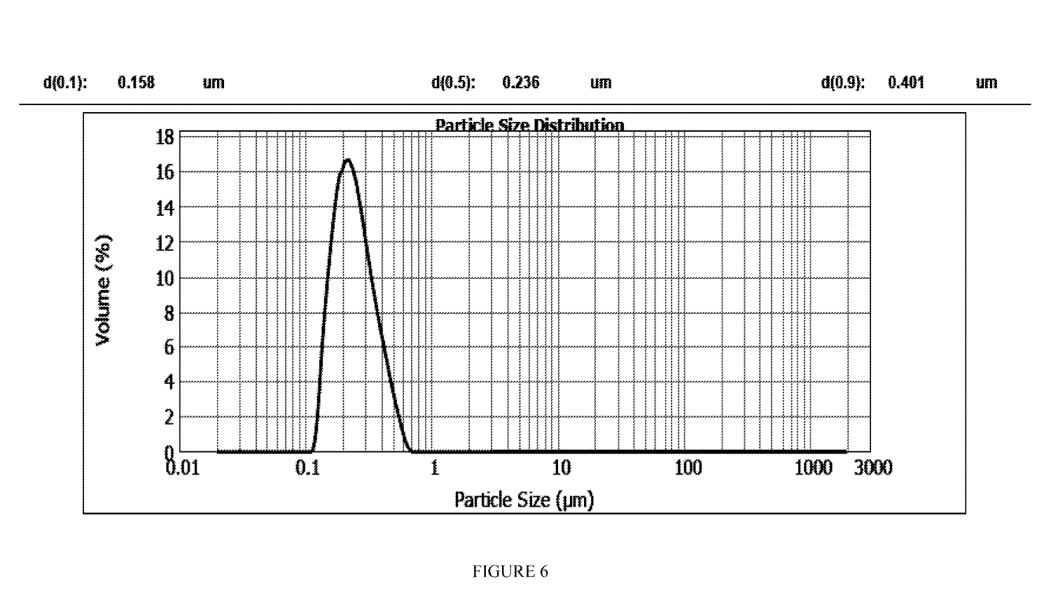

After being autoclaved 3× at 121° C. for 15 minutes, such a composition had a d(0.1) of 0.158 μm, a d(0.5) of 0.236 μm, and a d(0.9) of 0.401 μm. Uniformity was 0.316. D[3,2] was 0.231 μm. D[4,3] was 0.260 μm. Absorption was 0.1. The particle size distribution is shown in FIG. 6.

When different batches of emulsions were prepared, some variability was observed in the particle size distributions (data not shown). Overall, the results demonstrate the general stability of the composition with respect to particle size distribution when subjected to heat sterilization conditions of different degrees of harshness (e.g., 1×, 2×, or 3× autoclaving).

### Example 15

The following composition was prepared and analyzed. Glycerol was adjusted as needed to maintain osmolarity between 300-450 mosmols. The composition was passed through a M-110P microfluidizer (Microfluidics) five times and autoclaved three times (3×) at 121° C. for 15 minutes.

| Component | % w/w |
|---|---|
| Perfluorodecalin (cGMP) | 50 |
| Lipoid E80 | 4 |
| Tween 20 | 3 |
| Sodium EDTA | 0.01 |
| Glycerol—Adjust as needed | 3 |
| Soybean oil | 2 |
| Glycine | 0.06 |
| Water for Injection | 37.93 |
| pH adjustment with NaOH or HCl, 0.01N | pH to 8.4 |

Particle sizes were analyzed by laser diffraction using a Mastersizer 2000 (Malvern). The particle size distributions are expressed as volume distributions. d(0.1) was 0.161 μm; d(0.5) was 250 μm; and d(0.9) was 0.424 μm. Uniformity was 0.322. D[3,2] was 0.241 μm. D[4,3] was 0.274 μm. Absorption was 0.1.

### Example 16

### Method of Preparation of Emulsion Compositions

The following method was used to prepare certain compositions according to embodiments of the invention.

1. Add the Water for Injection (WFI) in a jacketed tank and heat up to 40° C.

2. Add sodium EDTA to WFI from Step 1 and stir to dissolve.

3. Add glycine to the solution from Step 2 and stir to dissolve.

4. Add glycerin to the solution from Step 3 and stir to dissolve. Adjust glycine as needed to maintain osmolarity between 300 and 450 mosmols.

5. Add Tween® 20 to the solution from Step 4 and stir to dissolve. Avoid excessive foaming.

6. Add Lipoid E80® to the solution from Step 5 and stir long enough to disperse the Lipoid E80® uniformly, yielding a milky white solution.

7. Add Soybean oil (Super refined Soyabean Oil USP=LQ-(MH), available from Croda Germany) to the solution from Step 6 and stir well to disperse the oil in small droplets.

8. Maintain the temperature of the vessel at 40° C.±5° C. and stir for 10-20 more minutes to form a uniform coarse emulsion.

9. Pass the product through a homogenizer and collect the homogenized emulsion in another tank. If necessary, dip the vertical homogenizer in the tank from Step 8 and homogenize for a sufficient time to distribute lipoid E80 uniformly. Keep the tank closed throughout to prevent the evaporation of water.

10. Add perfluorodecalin (cGMP product, available from Fluoromed) to the emulsion from Step 9 and stir for 10-20 minutes to form a coarse emulsion. Maintain the temperature of the product at 40° C. throughout.

11. Pass the product through the microfluidizer four times at 27,000 psi. Analyze the product for particle size distribution after every pass. Stir the product continuously throughout the operation. Also, keep the tanks covered to avoid evaporation of PFD and water.

12. Measure the pH of the product. If necessary, add sufficient amount of 0.01N NaOH or 0.01N of HCl to adjust the pH to 8.4. If the initial pH is very different than 8.4, use 0.1N NaOH or 0.1N HCl instead.

13. Pass the product from Step 12 one time through a M-110P microfluidizer (Microfluidics) at 27,000 psi and check the pH.

14. Send a sample for the determination of particle size distribution.

15. Fill the product in the vials/bottles and sterilize it by autoclaving.

16. Send another sample for the determination of particle size distribution.

17. The product turns white from the pre-autoclaved translucent appearance.

The target D90 value is about 260 to 270 nm after autoclaving for one time.

We claim:

1. A composition comprising an emulsion comprising:
particles of at least one perfluorocarbon; and
at least one emulsifying agent;
wherein the particles have an average particle size of between about 0.1 μm and about 5 μm.

2. The composition of claim 1, wherein the perfluorocarbon is perfluorodecalin, wherein the perfluorodecalin is present in an amount of about 50% by weight of the composition.

3. (canceled)

4. The composition of claim 1, wherein the at least one emulsifying agent comprises from about 1% to about 10% by weight of the composition.

5. The composition of claim 1, further comprising a component that is not significantly water soluble.

6. (canceled)

7. (canceled)

8. (canceled)

9. (canceled)

10. The composition of claim 1, wherein about 95% of the particles have an average particle size of less than about 1.5 μm.

11. The composition of claim 1, wherein the particles have a monomodal particle size distribution.

12. The composition of claim 1, wherein the particles have an average particle size of less than about 0.2 μm.

13. (canceled)

14. (canceled)

15. The composition of claim 1, wherein the composition has a shelf stability of at least 12 months at about 25° C.

16. The composition of claim 1, wherein the at least one emulsifying agent comprises one or more surfactants.

17. The composition of claim 16, wherein the one or more surfactants are present in an amount of between about 4% and about 8% by weight of the composition.

18. The composition of claim 16, wherein the one or more surfactants comprises a member selected from the group consisting of egg yolk phospholipids, soya phospholipids, soy lecithin, phosphatidylcholine, hydrogenated phosphatidylcholine, lysophosphatidylcholine, phosphatidylethanolamine, phosphatidylserine, phosphatidylinositol, phosphanolipids, phosphatidic acid, and mixtures thereof.

19. (canceled)

20. (canceled)

21. (canceled)

22. The composition of claim 1, wherein at least 90% of the total amount by volume of the particles have a size of less than about 0.3 μm.

23. The composition of claim 1, wherein at least 50% of the total amount by volume of the particles have a size of less than about 0.15 μm.

24. (canceled)

25. A method for producing a composition comprising an emulsion, comprising:
producing a surfactant dispersion in a water-salt medium; and
homogenizing at least one perfluorocarbon compound in the surfactant dispersion, wherein the resulting composition comprises an emulsion.

26. The method of claim 25, wherein the surfactant dispersion in the water-salt medium is produced by homogenization at a pressure of at least about 200 bar.

27. The method of claim 25 wherein the surfactant comprises a phospholipid.

28. The method of claim 25 further comprising heat sterilization of the resulting composition.

29. A formulation comprising a complex of the composition of claim 1 and oxygen-17 gas, wherein the oxygen-17 gas comprises from about 40% to about 90% saturation of the emulsion.

30. (canceled)

31. A method for preparing the formulation of claim 29 comprising:
(a) placing the composition of claim 1 into an oxygenation loading device;
(b) expelling the composition from the oxygenation loading device into an oxygenator device, wherein the oxygenator device comprises a plurality of hollow fibers and/or at least one over the dispersion disc encased within a larger container, the membranes of the hollow fibers and/or disc defining an intracapillary space within the hollow fibers and/or disc and an extracapillary space outside the hollow fiber and/or disc;
(c) exposing the composition to 170 gas by circulating the composition through the intracapillary space, wherein the 170 gas remains under positive pressure in the extracapillary space;
(d) allowing the composition to draw the 170 gas across the hollow fiber membrane and/or disc;
(e) binding the 170 gas with the composition within the intracapillary space to form a complex; and
(f) extracting the complex from the intracapillary space into a sealed, sterile container, wherein the complex remains under positive pressure.

32. (canceled)

33. (canceled)

34. (canceled)

35. (canceled)

36. (canceled)

37. (canceled)

38. (canceled)

39. (canceled)

40. (canceled)

41. A method for preparing the formulation of claim 30 comprising:
(a) placing the composition of claim 1 into an oxygenation loading device;
(b) expelling the composition from the oxygenation loading device into an oxygenator device, wherein the oxygenator device comprises a plurality of hollow fibers and/or at least one over the dispersion disc encased within a larger container, the membranes of the hollow fibers and/or disc defining an intracapillary space within the hollow fibers and/or disc and an extracapillary space outside the hollow fiber and/or disc;
(c) exposing the composition to 170 gas by circulating the composition through the intracapillary space, wherein the 170 gas remains under positive pressure in the extracapillary space;
(d) allowing the composition to draw the 170 gas across the hollow fiber membrane and/or disc;
(e) binding the 170 gas with the composition within the intracapillary space to form a complex; and
(f) extracting the complex from the intracapillary space into a sealed, sterile container, wherein the complex remains under positive pressure.
